SEQUENCE LISTING (73 sequences)

SEQ ID NO: 1 - protein, 6 aa, synthetic construct
  REGION 1..6: note = Description of Artificial Sequence: Synthetic peptide
KKKGGG

SEQ ID NO: 2 - protein, 523 aa, synthetic construct
  REGION 1..523: note = Description of Artificial Sequence: Synthetic polypeptide
MVPQALLFVPLLVFPLCFGKFPIYTIPDKLGPWSPIDIHHLSCPNNLVVEDEGCTNLSGFSYMELKVGYISAIKMNGFTCTGVVTEAETYTNFVGYVTTTFKRKHFRPTPDACRAAYNWKMAGDPRYEESLHNPYPDYHWLRTVKTTKESLVIISPSVADLDPYDRSLHSRVFPGGNCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPGQLVNLHDFRSDEIEHLVVEELVKKREECLDALESIMTTKSVSFRRLSHLRKLVPGFGKAYTIFNKTLMEADAHYKSVRTWNEIIPSKGCLRVGGRCHPHVNGVFFNGIILGPDGNVLIPEMQSSLLQQHMELLVSSVIPLMHPLADPSTVFKNGDEAEDFVEVHLPDVHERISGVDLGLPNWGKYVLLSAGALTALMLIIFLMTCWRRVNRSEPTQHNLRGTGREVSVTPQSGKIISSWESYKSGGETG

SEQ ID NO: 3 - protein, 101 aa, synthetic construct
  REGION 1..101: note = Description of Artificial Sequence: Synthetic polypeptide
ENPRLGMSCDIFTNSRGKRASKGSETCGFVDERGLYKSLKGACKLKLCGVLGLRLMDGTWVAMQTSNETKWCPPGQLVNLHDFRSDEIEHLVVEELVKKRE

SEQ ID NO: 4 - protein, 15 aa, Lyssavirus rabies
ACKLKLCGVLGLRLM

SEQ ID NO: 5 - protein, 15 aa, Lyssavirus rabies
KSVRTWNEIIPSKGC

SEQ ID NO: 6 - protein, 15 aa, Lyssavirus rabies
WCPPGQLVNLHDFRS

SEQ ID NO: 7 - protein, 15 aa, Human papillomavirus
RAHYNIVTFCCKCDS

SEQ ID NO: 8 - protein, 15 aa, Protoparvovirus carnivoran1
  source 1..15: note = Canine parvovirus
QPDGGQPAVRNERAT

SEQ ID NO: 9 - protein, 12 aa, Protoparvovirus carnivoran1
  source 1..12: note = Canine parvovirus
GAVQPDGGQPAV

SEQ ID NO: 10 - protein, 9 aa, Haemophilus influenzae
GDKTTFKQS

SEQ ID NO: 11 - protein, 15 aa, Clostridium tetani
QYIKANSKFIGITEL

SEQ ID NO: 12 - protein, 20 aa, Clostridium tetani
NSVDDALINSTKIYSYFPSV

SEQ ID NO: 13 - protein, 17 aa, Clostridium tetani
PGINGKAIHLVNNESSE

SEQ ID NO: 14 - protein, 15 aa, Canine morbillivirus
QKTNFFNPNREFDFR

SEQ ID NO: 15 - protein, 9 aa, Streptococcus pneumoniae
DRVPEEASR

SEQ ID NO: 16 - protein, 9 aa, Measles morbillivirus
KLWCRHFCV

SEQ ID NO: 17 - protein, 10 aa, Measles morbillivirus
FMYMSMLLGV

SEQ ID NO: 18 - protein, 10 aa, Measles morbillivirus
ILPGQDLQYV

SEQ ID NO: 19 - protein, 10 aa, Canine adenovirus 2
CYIIGNCAVL

SEQ ID NO: 20 - protein, 11 aa, Rotavirus sp.
  source 1..11: note = Rotavirus
DEMVRESQRNG

SEQ ID NO: 21 - protein, 11 aa, Mumps orthorubulavirus
KPRTSTPVTEF

SEQ ID NO: 22 - protein, 9 aa, Mumps orthorubulavirus
ALDQTDIRV

SEQ ID NO: 23 - protein, 9 aa, Canine parainfluenza virus
NTVEYFTSQ

SEQ ID NO: 24 - protein, 10 aa, Canine parainfluenza virus
GVTGLLTNAA

SEQ ID NO: 25 - protein, 9 aa, Canine parainfluenza virus
IPNPLLGLD

SEQ ID NO: 26 - protein, 14 aa, Influenza A virus
GLFGAIAGFIEGGW

SEQ ID NO: 27 - protein, 20 aa, Rubivirus rubellae
  source 1..20: note = Rubella virus
GLLACSAKCLYYLRGAIAPR

SEQ ID NO: 28 - protein, 17 aa, Rubivirus rubellae
  source 1..17: note = Rubella virus
GFLSGVGPMRLRHGADT

SEQ ID NO: 29 - protein, 27 aa, Rubivirus rubellae
  source 1..27: note = Rubella virus
APPTLPQPPRAHGQHYGHHHHQLPFLG

SEQ ID NO: 30 - protein, 12 aa, Rubivirus rubellae
  source 1..12: note = Rubella virus
TRWHRLLRMPVR

SEQ ID NO: 31 - protein, 6 aa, Hepatitis A virus
ELDKWA

SEQ ID NO: 32 - protein, 9 aa, Neisseria meningitidis
PNTRYRTPN

SEQ ID NO: 33 - protein, 9 aa, Human alphaherpesvirus 3
ILIEGIFFV

SEQ ID NO: 34 - protein, 9 aa, Human alphaherpesvirus 3
NIQPGYRSI

SEQ ID NO: 35 - protein, 10 aa, Human alphaherpesvirus 3
SLPRSRTPII

SEQ ID NO: 36 - protein, 10 aa, Hepatitis B virus
SSWAFARFLW

SEQ ID NO: 37 - protein, 20 aa, Mycobacterium tuberculosis
NVTSIHSLLDEGKQSLTKLA

SEQ ID NO: 38 - protein, 15 aa, Poliovirus 1
TTHIEQKALAQGLGQ

SEQ ID NO: 39 - protein, 18 aa, Clostridium tetani
FIGITELKKLESKINKVF

SEQ ID NO: 40 - protein, 11 aa, Severe acute respiratory syndrome coronavirus 2
SFIEDLLFNKV

SEQ ID NO: 41 - protein, 15 aa, Lyssavirus rabies
IFTNSRGKRASKGSE

SEQ ID NO: 42 - protein, 15 aa, Lyssavirus rabies
SKGSETCGFVDERGL

SEQ ID NO: 43 - protein, 15 aa, Lyssavirus rabies
DERGLYKSLKGACKL

SEQ ID NO: 44 - protein, 15 aa, Lyssavirus rabies
GACKLKLCGVLGLRL

SEQ ID NO: 45 - protein, 15 aa, Lyssavirus rabies
LGLRLMDGTWVAMQT

SEQ ID NO: 46 - protein, 15 aa, Lyssavirus rabies
VAMQTSNETKWCPPG

SEQ ID NO: 47 - protein, 15 aa, Lyssavirus rabies
HDFRSDEIEHLVVEE

SEQ ID NO: 48 - protein, 15 aa, Lyssavirus rabies
LVVEELVKKREECLD

SEQ ID NO: 49 - protein, 15 aa, Lyssavirus rabies
ENPRLGMSCDIFTNS

SEQ ID NO: 50 - protein, 9 aa, Lyssavirus rabies
GCTNLSGFS

SEQ ID NO: 51 - protein, 6 aa, Lyssavirus rabies
KLCGVL

SEQ ID NO: 52 - protein, 4 aa, Lyssavirus rabies
HDFH

SEQ ID NO: 53 - protein, 9 aa, Lyssavirus rabies
KSVRTWNEI

SEQ ID NO: 54 - protein, 18 aa, Canine morbillivirus
SHGMGIVAGSTGATQSAL

SEQ ID NO: 55 - protein, 18 aa, Canine morbillivirus
MTAAQITAGIALHQSNLN

SEQ ID NO: 56 - protein, 19 aa, Protoparvovirus carnivoran1
MSDGAVQPDGGQPAVRNER

SEQ ID NO: 57 - protein, 18 aa, Canine adenovirus 2
GWSFTRIKQRETPALGSP

SEQ ID NO: 58 - protein, 12 aa, Canine morbillivirus
SHGMGIVAGSTN

SEQ ID NO: 59 - protein, 11 aa, Canine morbillivirus
GPSVSAENVRQ

SEQ ID NO: 60 - protein, 11 aa, Canine morbillivirus
INPELRPIIGR

SEQ ID NO: 61 - protein, 9 aa, Canine morbillivirus
TQSALKSTG

SEQ ID NO: 62 - protein, 18 aa, Canine morbillivirus
MTAAQITAGIALHQSNLN

SEQ ID NO: 63 - protein, 12 aa, Canine morbillivirus
GDKYPIHFNDER

SEQ ID NO: 64 - protein, 15 aa, Canine morbillivirus
QKTNFFNPNREFDFR

SEQ ID NO: 65 - protein, 9 aa, Canine morbillivirus
ARGDIFPPY

SEQ ID NO: 66 - protein, 22 aa, Canine morbillivirus
MSDGAVQPDGGQPAVRNERATG

SEQ ID NO: 67 - protein, 15 aa, Canine morbillivirus
KTAVNGNMALDDTHA

SEQ ID NO: 68 - protein, 27 aa, Canine morbillivirus
ALGLPPFLNSLPQSEGGTNFGYIGVQQ

SEQ ID NO: 69 - protein, 14 aa, Protoparvovirus carnivoran1
PKIFINLAKKKKAG

SEQ ID NO: 70 - protein, 13 aa, Protoparvovirus carnivoran1
GQPAVRNERATGS

SEQ ID NO: 71 - protein, 17 aa, Protoparvovirus carnivoran1
DGAVQPDGGQPAVRNER

SEQ ID NO: 72 - protein, 18 aa, Protoparvovirus carnivoran1
NSLPQSEGATNFGDIGVP

SEQ ID NO: 73 - protein, 17 aa, Protoparvovirus carnivoran1
GKRNTVLFHGPASTKGK

(19) United States
(12) Patent Application Publication
(10) Pub. No.: US 2025/0057940 A1
(43) Pub. Date: Feb. 20, 2025

(54) IMMUNE MEMORY ENHANCED PREPARATIONS AND USES THEREOF

(71) Applicant: TORIGEN PHARMACEUTICALS, INC., Farmington, CT (US)

(72) Inventors: Mark Suckow, Lexington, KY (US); Ashley Kalinauskas, Vernon, CT (US); Ryan Michael Clauson, Wethersfield, CT (US)

(21) Appl. No.: 18/752,524

(22) Filed: Jun. 24, 2024

Related U.S. Application Data
(63) Continuation of application No. 17/460,026, filed on Aug. 27, 2021, now abandoned.
(60) Provisional application No. 63/072,073, filed on Aug. 28, 2020.

(51) Int. Cl. A61K 39/215; A61K 39/00; A61K 39/39

(52) U.S. Cl. CPC A61K 39/215; A61K 39/39; A61K 2039/552

(57) ABSTRACT

Formulations and preparations having immune memory enhanced properties are disclosed that provide for enhancing immune response against a tumor growth, cancer, infectious agent, bacteria, virus or other infectious or non-infectious agent. The vaccine formulation includes an immune memory invoking component, such as an antigen of an infectious agent, virus (e.g., Rabies), bacteria, prion, neo-antigen or other moiety antigen, and a targeted antigen (e.g., a harvested tumor tissue (B-cell, T-cell, epitopes)). The vaccine formulation/preparations may comprise a target infectious agent protein/peptide component (such as a SARS-Cov-2 spike protein epitope) mixed with, or fused to (or otherwise conjugated) an immune-memory associated viral antigen (such as Rabies, polio, or other peptide/protein antigen or peptide or fragment thereof).

### CROSS-REFERENCE TO RELATED APPLICATIONS

This application is a continuation application of U.S. patent application Ser. No. 17/460,026, filed Aug. 17, 2021, which claimed priority to U.S. Provisional Patent Application No. 63/072,073, filed Aug. 28, 2020, the disclosure of each of which is incorporated herein by reference in its entirety.

### SEQUENCE LISTING

The instant application contains a Sequence Listing which has been submitted electronically in WIPO ST 26 format and is hereby incorporated by reference in its entirety. Said WIPO ST 26 copy, created on Oct. 18, 2024, is named 119601-000016 SL.xml and is 67,669 bytes in size.

### FIELD OF THE INVENTION

The present invention relates to the field of immuno-therapy and immuno-prophylaxis, particularly to immuno-stimulatory preparations that provide and/or invoke an enhanced immune-memory associated response.

### BACKGROUND OF THE INVENTION

Tumor immunotherapy has revolutionized cancer treatment. However, current therapies remain suboptimal and are often not effective in some subjects. Exhaustion of tumor-specific T cells challenges the effectiveness of these approaches, and check point blockade therapies aimed at reversing exhaustion, have met with success in specific subsets of subjects, with some becoming refractory to these treatments.

Immunological memory is a potent mechanism by which conventional infectious agent vaccines establish life-long immunity in a subject. The establishment of immunological memory in a subject means that upon re-exposure to the pathogen, a highly specific and rapid lymphocyte reaction will launch. This lymphocyte reaction can be in the form of memory B-cell differentiation to plasma cells for production of antibodies, or via memory T-cell differentiation to effector cells for cytotoxic cell killing.

Subjects in whom current immunotherapies are not efficacious have tumors that have little inflammation and T-cell infiltration, and are called immunologically “cold” tumors. One approach examined for treating “cold” tumors is with in-situ vaccinations. These vaccinations provide for alpha-gal lipids delivered intratumorally to the tumor, and results in the incorporation of these lipids into the cell membrane of the cells. A number of viral vaccines have been delivered intratumorally to drive immunogenicity using this approach, through vaccine memory and de novo responses. The application of these approaches has been limited to the repurposing of established vaccines. Few examples of even these types of vaccine-specific epitopes (protein, peptides, carbohydrates) have been described.

The medical arts remain in need of more effective, robust treatments and preparations that employ a subjects' existing immune memory mechanisms. A solution to the long felt need of patient populations refractory to available treatments is also lacking.

### SUMMARY OF THE INVENTION

The present invention meets the above and other important medical needs in the art.

In one aspect, an immune response enhancing preparation is provided. In some embodiments, the preparation comprises a first component comprising an anti-cancer agent (moiety) and a second component comprising an immune memory invoking component. In another aspect, the preparation comprises a first component comprising an anti-infectious disease agent (moiety) and a second component comprising an immune memory invoking component. In some aspects, the immune memory invoking component comprises an antigen component of an infectious agent (viral, bacterial, parasitic, a prion). Thus, the immune memory invoking component comprises a moiety associated with an infectious agent, a tumor, a cancer, a moiety that is associated with a physiologically detectable response to a condition resulting from a prior exposure to an infectious disease, or a moiety that is not associated with an infectious agent at all.

In alternative embodiments, the immune memory invoking component may comprise an immunogenic component such as a protein, peptide, carbohydrate, glycoprotein or any combination thereof.

The anti-cancer component may comprise an inactivated tumor tissue cell preparation. In some cases, the inactivated tumor tissue cell preparation may comprise an autologous tumor cell preparation or an non-autologous tumor cell preparation. The term inactivated is used to describe a preparation which is essentially free of malignant tumor and/or cancer cells and is not otherwise infectious.

In the some embodiments, a base antigen immunization may be provided. Administration of the base antigen immunization could be provided before (prior to) administering an immune-memory enhancing preparation to the subject. This base antigen immunization may be provided any time before, just before, or even simultaneously with the administration of the immune memory enhancing preparations. Such would provide a novel platform providing an overall immune-memory enhanced health protective regimen for a subject, such as in providing an improved and/or enhanced response to a schedule of vaccinations in a subject.

A subject's prior exposure to an antigen, such as occurs when a subject is vaccinated against a viral, bacterial, parasitic, fungal or with non-infectious immunogens, or the subject is otherwise exposed and/or comes in contact with a particular infectious and/or immuno-stimulatory agent (such as may occur naturally), results in an immune memory being developed in the subject's system. This immune memory is developed particularly in the subjects population of immune cells. When the subject is subsequently exposed to an antigen of the infectious agent, the immune system of the subject is stimulated at an enhanced level (compared to the level of immune cell activation in a subject that had not been prior exposed to the infectious agent), thus enabling the subject to mount a more rapid and more robust response against the infectious agent. Association of a component of the infectious agent (such as a peptide) with a second component, such as an anti-cancer or other target component, has been discovered in the present studies to invoke a more robust general state of immuno-activation in the subject, serving to enhance attack, inhibit, and/or halt or retard the growth of, a tumor and/or cancer in the subject so treated.

The methods and compositions presented here function to activate a subject's own immune system by employing at least one component (viral, bacterial, non-viral, non-bacterial, synthetic, fungal, prion, non-infectious agent associated moiety or other natural or non-naturally occurring protein and/or peptide) associated with an existing immune-memory sensitivity in the subject. This immunological response phenomenon is harnessed to enhance a subjects immune response against an identified tumor and/or cancer in the subject by including a selected second component, such as an antigen associated with a tumor, cancer, or other condition, in the preparation. The subject's overall activated immune system response to the first component thus functions to generally alert the subject's immune response mechanisms, and results in improved immuno-surveillance, and the successful detection and attack of any other foreign moiety (e.g., a tumor or cancer) and/or disease causing entity that may be present in the subject. These other foreign moieties often times go undetected and/or blocked from immune surveillance in a subject, escaping the subject's own protective physiological response. Such is characteristic of subjects observed to have become refractory to treatment.

The subject's immune memory response to a viral, bacterial or other antigenic component thus enhances the subjects' immune response to a cancer and/or tumor antigen present.

The present preparations and methods therefore effectively eliminate and/or reduce failure of a subjects own immune surveillance to detect and respond to a foreign body, such as a cancer or tumor. The failure of a subjects own immune surveillance to detect a particular cancer or tumor, for example, may be the result of existing suppression mechanisms in the subject. With the dual immuno-antigen stimulating approach, a cancer or tumor will not escape the subject's immune surveillance, because of the immuno-memory stimulation effect provided by the bacterial, parasitic, viral or other immuno-memory stimulatory antigen component (that may or may not be part of or associated with an infectious agent) in the treatment preparation.

In some embodiments, an immune memory enhancing preparation is provided. In some embodiments, the preparation comprises a cancer and/or tumor antigen that is associated, decorated, or otherwise provided with, a second component that augments (enhances) immune response in a subject, This second component, for example, may comprise a peptide, protein, synthetic, non-synthetic, or other moiety that is associated with an infectious disease, or disease causing bacteria, non-bacterial, viral (such as the infectious agent for rabies, tuberculosis, measles, tetanus, HPV, influenza, etc.), non-viral, fungal, non-fungal, or any combination of these.

The immune memory response to the second component of the preparation will stimulate immune system function in the treated subject, and mount a response against the first component (such as a cancer, tumor, cancer and/or tumor antigen) also included in the preparation. The preparations thus provide an immune-memory enhanced treatment and/or therapy to a subject. Such preparations and methods thereby provide alternative, effective treatment options for subjects that have become refractory or otherwise non-responsive to conventional treatments for specific cancers, tumors, infectious diseases, parasitic diseases, etc.

The preparations may comprise a whole-tissue suspension preparation, a tumor cell, a cancer cell, a bacterial cell, or parasitic organism associated antigen component, a neoantigen component, or any combination of these, or any component part, fragment or derivative moiety of these (i.e., protein, peptide, carbohydrate). The whole tissue suspension may be of an autologous or non-autologous tumor and/or cancer source (autologous, allogenic, xenogeneic). The whole tissue preparation is a deactivated tissue preparation, and may be deactivated by treatment with glutaraldehyde.

Advantages provided with the present invention include techniques and preparations that overcome the limitations associated with treating subjects that have become refractory to conventional cancer treatments. In some situations, these subjects may be described as having a “cold” tumor and/or cancer. Methods for treating a “cold” tumor and/or cancer are provided by administering a preparation as described herein either directly to the tumor and/or cancer site, or administering the preparation to a subject via a more general route (e.g., i.p., i.v., intramuscular, subcutaneous, intragastric, oral, intrathecal, intraperitoneal, intranodal, etc.) to the subject. In this manner, lymphocyte infiltration will be invoked in the subject, effectively and more quickly mobilizing lymphocytes to a cancer and/or tumor site. In this manner, the immune memory response may provide for stimulation of anti-tumor immunity against a “cold” tumor and/or cancer. While such may be accomplished by in-situ vaccination at the tumor site, this result can also be achieved through administering the preparation through other routes.

In some embodiments, the cancer and/or tumor vaccine may be further described as comprising a first component that is a cancer cell antigen, a tumor cell antigen, a whole tumor cell preparation, a whole cancer tissue preparation, or other antigen of interest associated with these, comprising a peptide, protein or peptide fragment, such as a peptide, protein or peptide fragment identified and/or associated with a cancer, tumor, or combination of target moieties. In some embodiments where the antigen is a cancer antigen, the antigen may comprises a specific selected antigen (e.g., an epitopic antigen) of the specific tumor or cancer identified in a subject, or of a tumor/cancer epitopic antigen (antigens) of a cancer/tumor having a wide-spread prevalence in a population of subjects.

The preparations and methods provide for treatment of a human and any animal, including canine, bovine, feline, avian, marine, porcine, equine, human or other vertebrate or invertebrate animal, including, for example, honeybees. For example, it is envisioned that the preparations and methods provided herein may be incorporated in honeybee and other medicines.

In some embodiments, the method would comprise providing a vaccine, such as a cancer and/or tumor or other infectious or pathogenic microorganism vaccine, to a subject.

An immune memory enhancing preparation, provided as a vaccine preparation for example, is disclosed as part of the present invention, that may be used in the practice of the described methods. The vaccine may comprise a first component comprising a target moiety of interest, and a second component capable of invoking an immune memory response in a subject. In some embodiments, the vaccine will comprise a second component that comprises an epitopic peptide or protein associated with an infectious agent, a virus, a bacterial agent or other immuno-memory response invoking moiety in the subject. The second component may comprise a moiety associated with an immune response that has developed in the subject naturally (i.e., without vaccination), or an antigen (protein, peptide, artificial, naturally-occurring) of a virus, bacteria, infectious microorganism or other agent associated with a disease or illness to which the subject has been immunized against (e.g., distemper, adenovirus-2, parainfluenza, parvovirus, tetanus, anthrax, rotavirus, polio, meningitis, HPV, influenza, rabies (e.g., rabies antigen for a canine subject that had been previously immunized for rabies, etc.)). The method provides for invoking an immune response in a subject to the first component (a target moiety of interest) that is enhanced (produces a greater immune response, and hence an improved protective effectiveness) in the presence of the second component in the animal, compared to immune response to the first component in the subject in the absence of the second component. In some embodiments, the second component comprises an epitopic peptide associated with an infectious organism, a virus, a bacterial agent, a parasitic organismo, a fungus, any other synthetic or naturally occurring (non-synthetic) equivalent thereof, or any combination of these. In some embodiments, the first component comprises a tissue cell preparation, and the second component is mixed with the tissue cell preparation. The tissue cell suspension may comprises tumor and/or cancer tissue that has been mechanically disrupted so as to form a fluid-like preparation containing the mechanically dispersed tumor tissue.

The tumor cell/tissue and/or cancer cell/tissue antigen as a first component of the preparations described herein may comprise an inactivated (non-malignant) autologous tissue preparation (for example, prepared from a tumor tissue harvested from a subject to which the preparation will be administered), a tissue lysate, tumor associated antigens (TAAs), neoantigen, or inactivated non-autologous tissue, or a combination of any or all of these, including a combination of an inactivated autologous tumor tissue and/or cancer tissue and an inactivated non-autologous tumor and/or cancer tissue. In addition, the non-autologous tissue component may be prepared from a naturally occurring tissue, such as a tissue obtained from an animal or human that will not be receiving the cancer vaccine. Alternatively, the non-autologous tissue component may be a synthetically produced tumor, tumor specific peptide, and/or cancer tissue. In this regard, it is envisioned that a synthetically produced tumor and/or cancer tissue may be prepared as a 3-dimensional tumor and/or cancer synthetic tissue, and provided in the presently described tissue vaccines and preparations. All components derived from a tumor or cancer preparation are inactivated The term “inactivated” is intended to mean unable to cause or create a cancer or tumor growth in any animal, and that is not malignant.

In some embodiments, a method of inhibiting, preventing, or enhancing inhibition of tumor growth or cancer growth or spread in a subject is provided. The method may comprise administering to the subject an immuno-enhancing preparation comprising a tumor and/or cancer antigen or peptide and a moiety capable of stimulating an immune memory response in the subject. The moiety capable of stimulating an immune memory response in the subject may comprise, for example, an antigenic epitope of a virus (such as a rabies virus antigen), or any other synthetic or naturally occurring moiety capable of stimulating an immune memory recognition response in the subject. The treatment method may provide for the administration of a personalized and/or customized preparation that includes specific antigens identified from a subject's tumor. The preparation may further comprise an adjuvant.

By way of example, a tissue component may comprise the present preparations, and may comprise a previously prepared autologous or non-autologous tumor tissue component. Such tissue components may be prepared ahead of time and made available to the veterinarian. physician, nurse, technical staff, qualified veterinary or medical care provider, or other technician or care giver, at the time a treatment is to be administered.

As noted, the preparations may or may not further comprise an adjuvant. By way of example, the adjuvant may comprise any number of materials, including SIS (particulate, gel. sheet), alum, TLR agonists (i.e., CpG oligonucleotides, monosphosphoryl lipid A. flagellin, cGAMO associated derivatives, Poly (I:C), imiquimod) and others.

An ECM may also be included in the preparations and formulations. An ECM is described in U.S. Pat. No. 8,062,646. The teachings of this patent are specifically incorporated herein by reference in its entirety.

In some aspects, the present invention may be used in methods for providing both or either of a therapeutic or prophylactic treatment for inhibiting, halting, reducing, the growth of progression of a cancer, tumor or other disease or infection in a subject. In some embodiments. the method comprises administering and/or providing the immune memory enhancing preparation to a subject. In some embodiments, the preparations may comprise a non-autologous or an autologous tissue component and a viral, bacterial, infectious agent, cancer, or tumor antigen. In some embodiments, the viral, bacterial, infectious agent, cancer or tumor antigen is a peptide or selected epitopic motif of the viral, bacterial, infectious agent, cancer or tumor antigen, and is associated with cells of the tissue component (such as by glutaraldehyde fixation), or that are provided as a suspension of the peptides and/or antigenic epitope components in the tissue preparation. Biological materials extracted and/or drained from lymph nodes may also provide a source of tumor antigens for use in the present preparations and methods. Such provides a rick and useful source for antigens, particularly for lymphoma.

Combination Therapeutic Treatment: Methods of treating and/or inhibiting a cancer or tumor are provided that employ a combination therapeutic regimen. In some embodiments, the method comprises administering the immune memory enhancing preparations described herein together with one or more conventional chemotherapeutic cancer treatment modalities, such as anti-cancer biological agents, radiation (radiotherapy), surgery, or any combination of these. The health care attendant/professional (for example, veterinarian, trained technical attendant, physician, laboratory technician, nurse) may, at the time of treatment and in monitoring the particular needs of the subject and/or patient, select which of these treatments, are best suited for the subject and/or that illicit a most beneficial response in the subject.

In some instances, a tumor from which an antigen may be identified and selected for use in the present immune memory enhancing preparations and methods of use may constitute a tumor or lymph node that is of veterinary or human origin, and may be selected from virtually any type of cancer type. In this regard, the tumor and/or cancer antigenic component of the preparation is found to be effective for enhancing immune response and inhibiting and/or retarding growth of virtually any cancer and/or tumor type in a subject, and is not required to be derived from the same tumor and/or cancer type to be treated in a subject.

In an alternative aspect, the immune memory enhancing preparations may comprise a neoantigen component.

The immune enhancing preparations may be formulated and delivered in a soluble form, conjugated to a carrier protein (i.e., streptavidin, KLH, BSA. etc.), conjugated to or encapsulated in/by MIM-SIS or other extracellular matrix formulation, or produced as an alternative nano-micro formulation, such as encapsulation in alginate chitosan, or other materials to be delivered by routes that include subcutaneous, intramuscular, intravenously, intranodally, intrathecally, intraperitoneally, orally, intratumorally, upon a resected tumor bed, or in an encapsulated form around or in the general vicinity of a tumor and/or cancer.

In some embodiments, the immune memory enhancing preparations may be provided as an intratumoral or around the perimeter of a tumor that has become non-responsive to treatment, and is determined to be immunologically “cold”. In this manner, an immunologically unresponsive tumor may be transformed to an immunologically responsive, or “hot” area by locally administering the preparations, and in this manner invoke the stimulation of a subject's immune cells, and promote the penetration of the subject's B-cell and T-cell populations to the tumor site. The resulting hot-pro-inflammatory environment in this manner functions to promote tumor clearance.

In some embodiments, excipients for use with the compositions disclosed herein include maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

The cancer vaccines are made to be compatible with a particular local, regional or systemic administration or delivery route. Thus, the vaccines may include carriers, diluents, or excipients suitable for administration by particular routes. Specific non-limiting examples of routes of administration for the vaccines are parenteral, e.g., intravenous, intra-arterial, intradermal, intramuscular, subcutaneous, intranodal, intrathecal, intratumoral, and other delivery types suitable for the treatment method or administration protocol.

In some embodiments, solutions or suspensions used for the parenteral application of a vaccine, include: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. In some embodiments, pH is adjusted with acids or bases, such as hydrochloric acid or sodium hydroxide.

The cancer vaccines and other preparations for injection may include sterile aqueous solutions (where water soluble) or dispersions and sterile powders for the extemporaneous preparation of sterile injectable solutions or dispersion. By way of example only, and not intending to be limited to those specific materials listed here, and as examples of those compositions prepared for intravenous administration, suitable carriers include physiological saline, bacteriostatic water, Cremophor ELTM (BASF, Parsippany, N.J.), or phosphate buffered saline (PBS). In some embodiments, the carrier is a solvent or dispersion medium containing, for example, water, ethanol, polyol (for example, glycerol, propylene glycol, and liquid polyethylene glycol, and the like), or suitable mixtures thereof. Fluidity is maintained, in some embodiments, for example, by the use of a coating such as lecithin, by the maintenance of the required particle size in the case of dispersion, and by the use of surfactants. Antibacterial and antifungal agents include, for example, parabens, chlorobutanol, phenol, ascorbic acid, and thimerosal. Isotonic agents, for example, sugars; polyalcohols such as mannitol or sorbitol; or sodium chloride, in some embodiments, are included in the composition. In some cases, also included may be an agent which delays absorption, in some embodiments, for example, aluminum monostearate or gelatin prolongs absorption of injectable compositions.

In some embodiments, the sterile injectable formulations of the cancer vaccine preparations are prepared by incorporating the cancer or other antigen in the required amount in an appropriate solvent with one or a combination of the above ingredients. Generally, dispersions are prepared by incorporating the active composition into a sterile vehicle containing a basic dispersion medium and any other ingredient. In the case of sterile powders for the preparation of sterile injectable solutions, methods of preparation include, for example, vacuum drying and freeze-drying which yields a powder of the active ingredient plus any additional desired ingredient from a previously prepared solution thereof.

The following terms are to be interpreted according to the following definitions in the description of the invention that follows:

Reference to “about” a value or parameter herein includes (and describes) variations that are directed to that value or parameter per se. For example, description referring to “about X” includes description of “X”.

The term “neoplastic” relates to a cell that divides more than it should or do not die when they should. Frequently, neoplastic cells form a mass of cells, referred to as a tumor. Neoplastic cells and neoplastic cell masses thereof may be benign (not cancer), precancerous, and/or malignant (cancer), and may be invasive, metastatic, non-invasive, or otherwise characterized.

The term “immunogen” refers to a moiety, which optionally can be administered to a subject, which induces an immunological response.

The terms “recipient”, “individual”, “subject”, “host”, “animal” and “patient”, are used interchangeably herein and in some cases, refer to any mammalian (human or non-human, including veterinary), non-mammalian, vertebrate, non-vertebrate subject for whom diagnosis, treatment, or therapy is desired. In particular, particularly animals include companion animals, such as canines.

The term “about X-Y” used herein has the same meaning as “about X to about Y.”

The use of the term “not” in description of a value or parameter generally means and describes “other than” a value or parameter. For example, the method is not used to treat cancer of type X means the method is used to treat cancer of types other than X.

The singular forms “a,” “or,” and “the” include plural referents unless the context clearly dictates otherwise.

The terms “treatment” or “treating” is used to refer to an approach for obtaining beneficial or desired results including clinical results. For purposes of this invention, beneficial or desired clinical results include, but are not limited to, one or more of the following: prophylactically protecting a subject against a disease (e.g., against developing cancer and/or a tumor), decreasing one more symptoms resulting from the disease, diminishing the extent of the disease, stabilizing the disease (e.g., preventing or delaying the worsening of the disease), preventing or delaying the spread (e.g., metastasis) of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, preventing/inhibiting a cancerous or tumorous growth or the rate of growth, and/or prolonging survival. Also encompassed by “treatment” is a reduction of pathological consequence of cancer. The methods of the invention contemplate any one or more of these aspects of treatment.

The term “individual” or “subject” is used synonymously herein to describe a mammal, poultry, birds, avian, marine (fish), porcine, including humans. An individual includes, but is not limited to, a human, bovine, porcine, feline, canine, murine, equine, bovine, marine, and any primate or mammal. In some embodiments, the subject is human. In some embodiments, an subject may be identified as suffering or having been identified/diagnoses to have a disease, such as a cancer and/or tumor. In some embodiments, the subject may be generally be identified as otherwise in need of treatment.

The term “pharmaceutically acceptable” or “pharmacologically compatible” is meant a material that is not biologically or otherwise undesirable, e.g., the material may be incorporated into a pharmaceutical composition administered to an individual without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration.

These and other aspects and advantages of the present invention will become apparent from the subsequent detailed description and the appended claims. It is to be understood that one, some, or all of the properties of the various embodiments described herein may be combined to form other embodiments of the present invention. These examples are not intended to be limiting, but instead exemplary, as used in the practice of the present methods and preparations given the teachings of the present disclosure by one of ordinary skill in the therapeutic medicine and/or oncology treatment arts.

### DESCRIPTION OF THE SEVERAL VIEWS OF THE DRAWINGS

FIG. 1—Flow chart showing sequence of steps for preparing and administering a memory-enhanced vaccine preparation to a subject, such as to a veterinary subject (canine). In the figure, the canine subject is previously vaccinated for Rabies. Any previously administered conventional vaccine administered to a subject will provide the immuno-memory advantage and/or enhancement of therapeutic benefit for reducing and/or inhibiting tumor growth upon treatment with a vaccine formulation that includes antigenic components (epitopes) of the tumor of interest (the “target” antigen) and antigenic components (epitopes) of an antigen to which the subject had been previously exposed and to which an immune response was elicited (for example, the vaccine formulation may include a Rabies antigen, such as B-cell epitope peptides derived from the Rabies virus glycoprotein and/or in combination with T-cell epitopes (CD-4)).

FIG. 2A-FIG. 2E. B16F Melanoma Anti-Tumor Efficiency. Individual tumor volume growth curves for B16F10 tumors subcutaneously implanted into the flank of C57BL/6J mice at 25,000 cells per mouse threated with Saline (n=16) (FIG. 2A), VetiVax™ (n=22) (FIG. 2B), RabiVax™ (n=7) (FIG. 2C), RabiVax™+Adjuvant (n=7) (FIG. 2D), Pooled Tumor Volume Growth Curves for B16-F10 tumors of the aforementioned treatments (FIG. 2E). statistical comparisons are based on one-way ANOVA, followed by post hoc Turkey's pairwise comparisons. The asterisks denote statistical significance at the level of *p less than 0.05. ANOVA, analysis of variance.

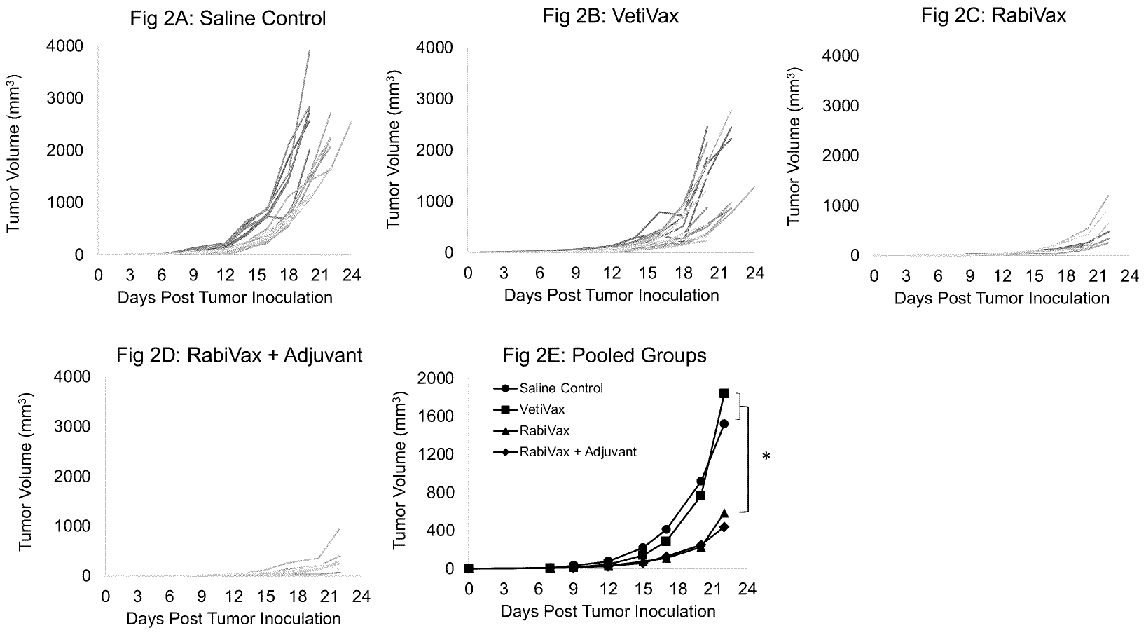

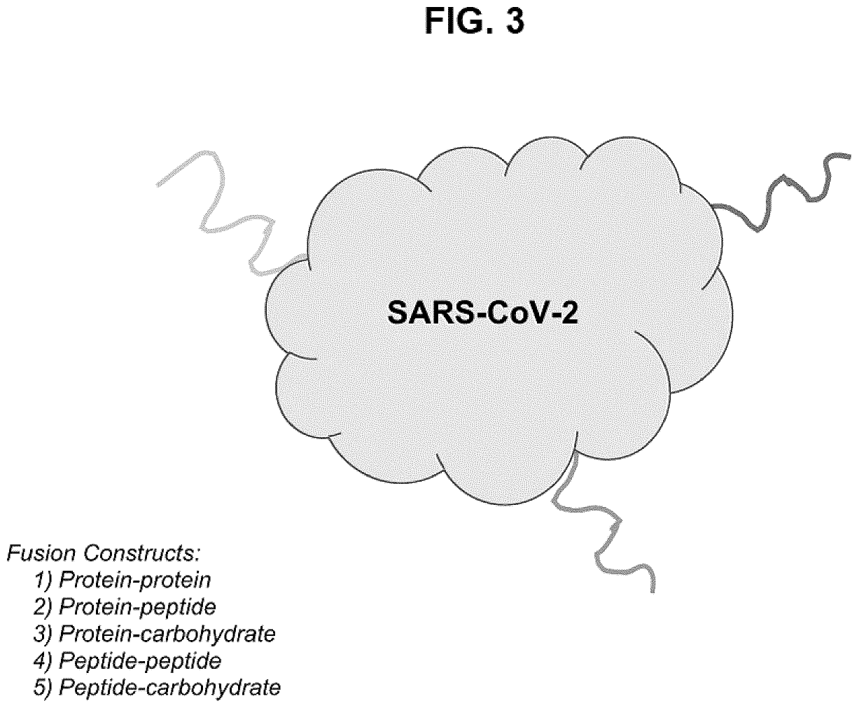

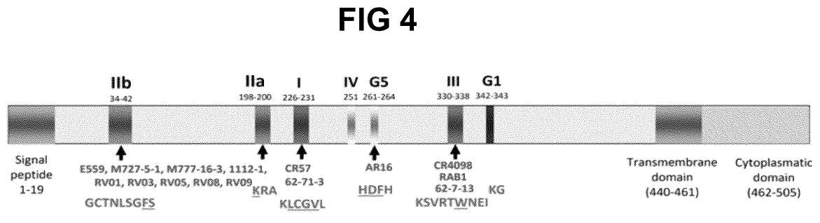

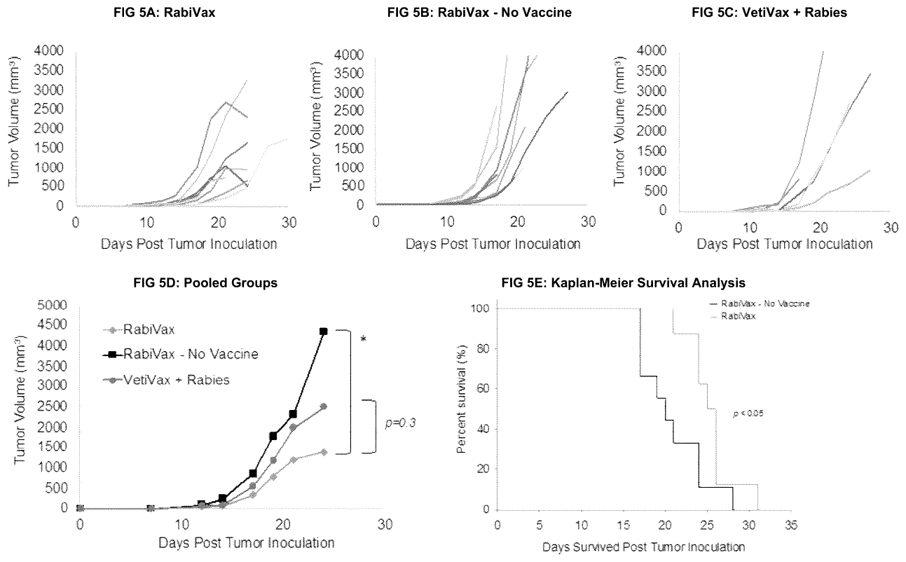

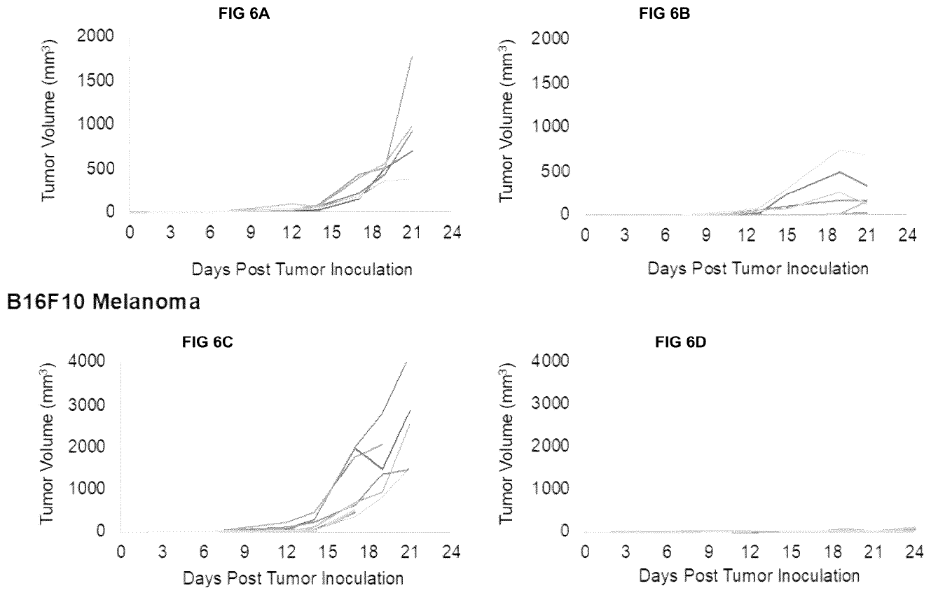

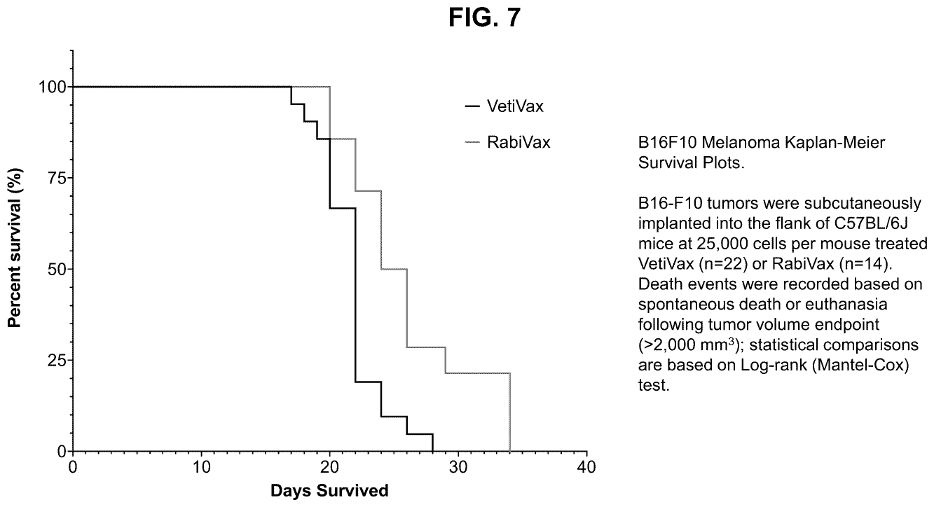

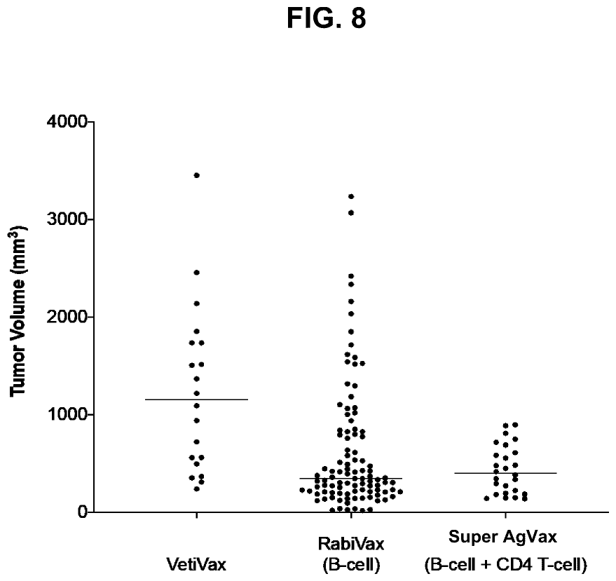

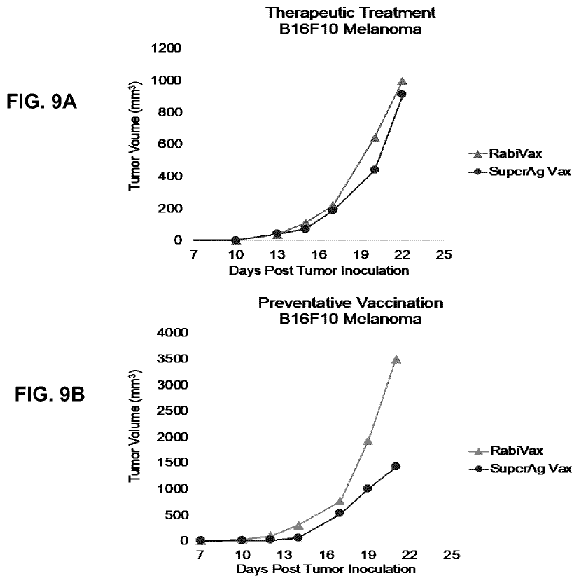

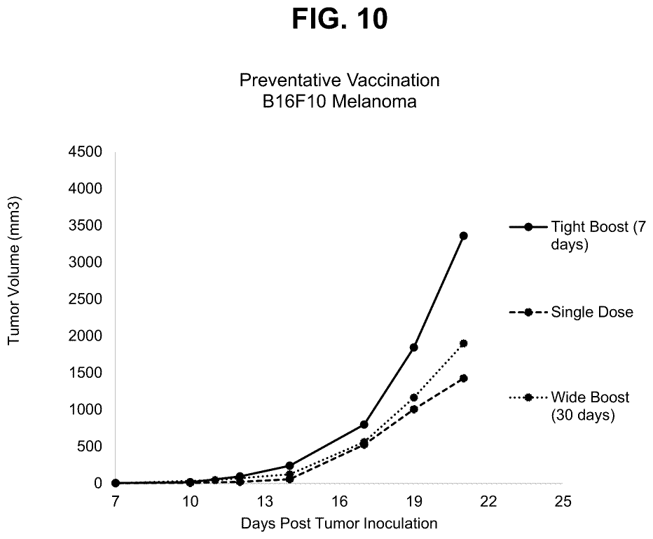

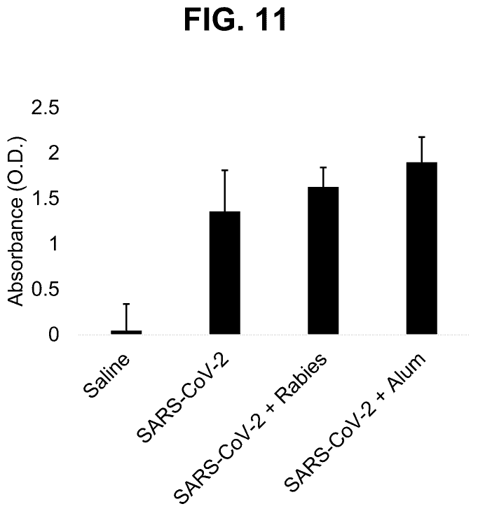

The adjuvant in the “RabiVax™+Adjuvant” group is CpG1826+MPLA, 10 ug each. Adjuvant was added as physical mixture. The data shows that the tumor size resulting from administration of a combination of rabies peptide plus the tumor tissue were much smaller than the tumor size resulting from administration of VetiVax™ (tumor tissue suspension) alone. The animals in the RabiVax™ treatment group and the RabiVax™+Adjuvant treatment group demonstrated significant tumor growth suppression. (solid circles=PBS (Control); solid squares=VetiVax™; solid triangles=RabiVax™. VetiVax™+5e6 GFT cells incubated/labeled/fixed to include a combination of 3 synthesized peptides. The 3 synthetic peptides were designed by isolating particular epitopic motifs selected from a rabies Pasteur protein sequence (FIG. 4). In FIG. 2F, B16-F10 tumors were subcutaneously implanted into the flank of C57BL/6J mice at 25,000 cells per mouse (VetiVax™ (n=22) or RabiVax™ (n=14)). Death events were recorded based on spontaneous death or euthanasia following tumor volume endpoint (<mm³); statistical comparisons are based on Log-rank (Mantel-Cox).

The data shows that the tumor size resulting from administration of a combination of rabies peptide plus the tumor tissue were much smaller than the tumor size resulting from administration of VetiVax™ (tumor tissue suspension) alone. The animals in the RabiVax™ treatment group and the RabiVax™+Adjuvant treatment group demonstrated significant tumor growth suppression. (solid circles=PBS (Control); solid squares=VetiVax™; solid triangles=RabiVax™. VetiVax™+5e6 GFT cells incubated/labeled/fixed to include a combination of 3 synthesized peptides. The 3 synthetic peptides were designed by isolating particular epitopic motifs selected from a rabies Pasteur protein sequence (See Example 8). In FIG. 2F, B16-F10 tumors were subcutaneously implanted into the flank of C57Bl mice at 25,000 cells per mouse (VetiVax™ (n=22) or RabiVax™ (n=14).

FIG. 3—Fusion Protein Construct Model for SARS (COVID19) vaccine. The Fusion Protein Construct may comprise a Protein-Protein, a Protein-Peptide, a Protein-Carbohydrate, a Peptide-Peptide, or a Peptide-Carbohydrate construct.

FIG. 4—Schematic representation of the RV glycoprotein. Major antigenic sites and their amino acid positions are shown above the bar. Arrows indicate MAb epitopes within antigenic sites. Figure discloses SEQ ID NOS 50-53, respectively, in order of appearance.

FIG. 5A-FIG. 5E—B16F Melanoma anti-tumor efficiency rabies vaccination and peptide conjugation dependency. Individual tumor volume growth curves for B16-F10 tumors subcutaneously implanted into the flank of C57BL/6J mice at 25,000 cells per mouse treated. Mice were treated when mean tumor volumes reached between 50-100 mm³ (day 12). Mice were treated 3 times at 5 day intervals. FIG. 5A—RabiVax™ with prior vaccination against rabies (n=8); FIG. 5B—RabiVax™ without prior vaccination against rabies (n=9); FIG. 5C—VetiVax™ plus rabies peptide as a physical mixture with prior vaccination against rabies (n=5). FIG. 5D—Pooled tumor volume growth curves for B16-F10 tumors of the aforementioned treatments. Statistical comparisons are based on one-way ANOVA, followed by post hoc Turkey's pairwise comparisons. The asterisks denote statistical significance at the level of p<0.05.

FIG. 6A-FIG. 6D—Prophylactic anti-tumor efficacy. Rabies vaccinated mice were treated three times at weekly intervals. CT26 colon cancer mice were treated with Saline (n=5) (FIG 6A), RabiVax™ (n=7) (FIG. 6B), prior to being inoculated with 5e6 total cells in the SC flank. B16-F10 melanoma mice were treated with Saline (n=6) (FIG. 6C) and RabiVax™ (n=6) (FIG. 6D), prior to being inoculated with 2.5e4 total cells in the SC flank. (NobiVAC-3 Term is used interchangeably with RabiVax™).

FIG. 7—B16F10 Melanoma Kaplan-Meier Survival Plots. B16-F10 tumors were subcutaneously implanted into the flank of C57BL/6J mice at 25,000 cells per mouse treated VetiVax™ (n=22) or RabiVax™ (n=14). Death events were recorded based on spontaneous death or euthanasia following tumor volume endpoint. (<2,000 mm³); statistical comparisons are based on Log-rank (Mantel-Cox) test. RabiVax™—no vaccine=dotted line; RabiVax™M=hatched line. The data show that a larger group of animals survived upon treatment with RabiVax™ (compared to no treatment), and that the survival time was enhanced in those animals treated with the RabiVax™ preparation (compared to no treatment). Improved survival in animals treated with the RabiVax™ is shown up to 34 days (21.4% of the animals treated with RabiVax™ survived to 34 days), while only 4.5% of the VetiVax™ treated animals survived even to 28 days.

FIG. 8—Tumor Volume in Tumor-Bearing Subjects-Day-20 post Inoculation with vaccine formulations, VetiVax™, RabiVax™, or SuperAg Vax RabiVax™. The scatter plots illustrate tumor volume pooled sample data sets from tumor bearing subjects treated with one of the three vaccine formulations.

FIGS. 9A 9B—Therapeutic and Prophylactic Efficacy of Immuno-Memory Autologous Therapy. The efficacy of the RabiVax™ and SuperAg Vax™ vaccine formulations was compared in FIG. 9A as a therapeutic, and in FIG. 9B as a prophylactic for inhibiting/preventing tumor growth, in vivo.

FIG. 10—Modification of Dosing-Interval Vaccine Boost Regimen on Efficacy of Immuno-Memory Autologous Vaccine Administration—RabiVax™ Vaccine Formulation Treatment.

FIG. 11—SARS-Cov-2 S1 Spike Protein Antigen—Vaccination Formulations. B-cell Epitope antigen vaccine formulation treatment in Rabies-vaccinated animals-Production of Spike protein specific anti-IgG antibodies. Rabies-vaccinated mice (n=5) were treated as described herein. These vaccinated mice were subsequently vaccinated (prime-boost) with S1 spike protein of SARS-COV-2 either: (1) SARS-Cov-2 alone, (2) SARS-COV-2 chemically cross-linked/conjugated to Rabies virus glycoprotein B-cell epitope peptides or (3) SARS-COV-2 physically mixed with Alum. Blood was collected from all mice via submandibular puncture at weekly intervals. S1 spike protein specific anti-IgG was determined by ELISA from blood from each group collected two-weeks post-boost (week 5).

### DETAILED DESCRIPTION

The following examples present a description of various specific aspects of the intended invention, and are not presented to limit the intended invention in any way.

### Example 1—Tumor Tissue Vaccination Protocol—Unique Epitope Peptides Associated with Rabies Immuno-Memory Response

The present example demonstrates the preparation of anti-tumor and/or anti-cancer tumor cell tissue preparations that are fixated to include immuno-memory recognized epitope peptides, proteins, etc. The selected immuno-memory recognized epitomic peptides elicit an immunological response in a subject that facilitates robust anti-tumor activity against a tumor and/or cancer in a subject in vivo, and thereby inhibits and/or reduces tumor growth.

In some embodiments, epitope antigens specific for rabies is fixated to the tumor tissue preparation to provide a tumor tissue vaccine preparation. This procedure will facilitate stimulation of the immunological memory already present in a rabies-vaccinated subject in vivo. By stimulating this existing immuno-memory against rabies in the subject, an immuno-response to the cancer-specific antigen or tumor will also be indirectly promoted, as a consequence of activating the subjects pre-conditioned immune cells targeting the rabies antigen.

### Procedure for Preparing a Tumor Tissue Cell Preparation

1. ) Tumor tissue is harvested from a donor animal. No formalin fixation of tumor tissue.
2. ) Tumor tissue is mechanically dissociated to create a tumor tissue cell suspension and/or preparation.
3. ) The tumor tissue cell suspension and/or preparation is chemically deactivated (glutaraldehyde treatment) to provide a deactivated tumor tissue cell preparation (deactivated=not capable of replicating, not malignant).
4. ) The deactivated tumor tissue cell tissue cell preparation is combined and/or incubated with a second component that comprises an immuno-memory stimulating peptide/protein/epitomic peptide antigen. This second component has a strong immuno-specific reactivity for an antigen of an infectious disease. Alternatively, the second component may comprise an immuno-memory stimulating peptide/protein/epitope peptide antigen specific for a particular cancer and/or tumor type identified in the subject being treated. The second component becomes cross-linked or fixed to the cells/suspension of the tumor tissue cell suspension.
5. ) The combined preparation may be further combined with an adjuvant (e.g., MIM-SIS adjuvant) to provide an adjuvanted preparation.

### Example 2—Preparation of Peptides for Anti-Cancer Tissue Preparations

The present example sets forth the method whereby antigenic components of an infectious disease-causing agent (virus, bacteria, etc.) may be identified and prepared that have strong immunoactivity-i.e., the ability to invoke a strong immuno-memory response in an animal. In this example, the infectious disease is rabies, and the infectious agent is the rabies virus. Peptides isolated for their immuno-memory invoking properties were selected, synthesized and used in the preparation of the inactivated tumor cell tissue suspension vaccine preparations.

The specific peptides created were independently selected by the present investigators based on internal selection criteria based on investigational experience and validation studies. A selection of literature was reviewed to examine B-cell and T-cell epitopes of the rabies virus glycoprotein. The present investigators paid specific focus to selected and particular linear B-cell epitopes. Once such particularly linear B-cell epitopes were selected, the sequences were further examined to identify potentially useful short peptide sequences within them. An initial screening was performed to evaluate immunogenic sequences of interest and to validate literature reports. From these studies, it was independently determined that a range of peptides between amino acids 230-260 would demonstrate the most robust immune memory response in dogs. This characteristic of the selected and synthesized non-naturally occurring peptides was determined by testing with a peptide specific serum IgG ELISA. The specific synthesized sequences were then tested with commercially available monoclonal antibodies directed against rabies virus glycoprotein. (Kuzmina, et al) and cross compare these binding results against binding reported with published B-cell epitopes. The present preparations and testing was not limited to a single epitope. Instead, a combination of many epitopes was examined, in a series of three-15 mers that can be conjugated to autologous cells via a KKKGGG-n-terminal peptide flanker sequence (SEQ ID NO: 1) via glutaraldehyde crosslinking.

```
(SEQ ID NO: 2)
                10 20 30 40
MVPQALLEVP LLVFPLCFGK FPIYTIPDKL GPWSPIDIHH
                50 60 70 80
LSCPNNLVVE DEGCTNLSGF SYMELKVGYI SAIKMNGFTC
                90 100 110 120
TGVVTEAETY TNFVGYVTTT FKRKHFRPTP DACRAAYNWK
              130 140 150 160
MAGDPRYEES LHNPYPDYHW LRTVKTTKES LVIISPSVAD
              170 180 190 200
LDPYDRSLHS RVFPGGNCSG VAVSSTYCST NHDYTIWMPE
              210 220 230 240
NPRLGMSCDI FTNSRGKRAS KGSETCGFVD ERGLYKSLKG
              250 260 270 280
ACKLKLCGVL GLRLMDGTWV AMQTSNETKW CPPGOLVNLH
              290 300 310 320
DERSDEIEHL VVEELVKKRE ECLDALESIM TTKSVSFRRL
              330 340 350 360
SHLRKLVPGF GKAYTIENKT LMEADAHYKS VRTWNEIIPS
              370 380 390 400
KGCLRVGGRC HPHVNGVFFN GIILGPDGNV LIPEMOSSLL
              410 420 430 440
QQHMELLVSS VIPLMHPLAD PSTVFKNGDE AEDFVEVHLP
              450 460 470 480
DVHERISGVD LGLPNWGKYV LLSAGALTAL MLIIFLMTCW
              490 500 510 520
RRVNRSEPTQ HNLRGTGREV SVTPQSGKII SSWESYKSGG
ETG
```

### Pasteur Strain—Extracellular Domain of Interest

(SEQ ID NO: 3)
ENPRLGMSCDIFTNSRGKRASKGSETCGFVDERGLYKSLKGACKLKLCG
VLGLRLMDGTWVAMQTSNETKWCPPGQLVNLHDFRSDEIEHLVVEELVK
KRE

Selection of Peptides: Peptide sequences were taken from the immunogenic regions of surface proteins on viral particles. These correspond with “B cell epitopes”, or targets that are primarily neutralized by antibody production from B cells. In viruses without defined B cell epitopes in literature, conserved sequences from better studied viruses were used instead. Being conserved across similar viruses indicates the potential for similar immunogenicity

Immune “Noise”. “Noise” is a term used to describe the immune system's ability to filter out “self” tissue from immunogenic “other” tissue. An autologous cancer vaccine by design creates “noise” as self-tissue is inevitably mixed in with tumor tissue

Central memory T cells are less prone to immunological exhaustion and self-tolerance. Tem cells are still affected by immunological noise, but not as dramatically as naive acutely stimulated T cells.

Theoretically, the Tem cells may be less prone to being affected by immunological noise, and their activation should result in increased cytotoxicity. IgG antibodies are more prone to opsonization than IgM antibodies. G Fc binds specifically to phagocytes, increasing antigen uptake and therefore presentation. Therefore, memory B cell responses should be more apt to cause increased opsonization and cross presentation, further increasing neoantigen presentation and uptake. The tumor cells being “tagged” by IgG may reduce “self” uptake and presentation, reducing the proportion of anergic lymphocytes.

Increased cross presentation and opsonization would therefore benefit by reducing the chance that “noise” from tissue would induce self-tolerance and prevent vaccine efficacy.

From the above trial and error selection process, and analysis in view of preliminary results achieved, the following 3 peptides were identified and synthesized for use in preparing the present preparations and vaccines. Peptides were custom synthesized by LifeTein.

The three isolated and selected peptides (N-terminal KKKGGG flanker sequence (SEQ ID NO: 1)+sequence) appear below

Rabies virus peptides:

(aa: 241-255)
(SEQ ID NO: 4)
ACKLKLCGVLGLRLM
(aa: 349-363)
(SEQ ID NO: 5)
KSVRTWNEIIPSKGC
(aa: 270-284)
(SEQ ID NO: 6)
WCPPGQLVNLHDFRS

The rabies peptides1—the peptides above were determined to represent immunologically strong epitopic peptides that were specific for rabies antigen. These three peptides were combined to provide a peptide cocktail, that included a combination of 50 μg each of 3 isolated peptides. As described above, the 3 unique epitopic peptides were identified and selected from the entire protein sequence for rabies of the Pasteur Rabies Strain.

RVG is commercially available. See Creative-diagnostics.com.

### Unique Epitopic Peptides

In this example, 3 unique peptides with selected epitopic peptide sequences having strong immunological activity for the infectious disease, rabies, were derived from the Pasteur

Rabies sequence (FIG. 4). Because rabies is an infectious disease against which animals are routinely vaccinated, it was envisioned that fixation of the tumor tissue cell preparation with the epitopic peptides selected would invoke immunological memory cell response in a subject, and thus enhance the anti-tumor promoting properties of the tumor tissue cell preparation in the subject.

It is to be understood that the creation and use of this particular model is not limited to rabies or to canines, or even to only cancer or veterinary animals. It is anticipated that the present protocols and teachings will be effective in non-cancer disease areas, such as infectious diseases, as well as for use in human subjects.

### Example 3—Inhibition of Tumor Growth—Vaccines With or Without Viral Peptide

The present example presents a comparative study of tumor growth in an animal vaccinated against an infectious disease (rabies), that is then treated with a deactivated tumor tissue cell preparation fixed to include one or more peptide epitopes specific for the infectious disease, verses tumor growth in an animal treated with a deactivated tumor tissue cell preparation that has not been fixed to include one or more peptide epitopes specific for the infectious disease. The study results are presented in FIG. 2.

FIG. 2 Description of Study: Mice (C57BL/6J mice) were vaccinated with a rabies vaccine. These vaccinated animals possess immuno-memory that will enhance the immuno-stimulation response of a subject upon re-exposure to rabies. These vaccinated animals were treated to grow a B16F10 tumor. To induce the growth of the tumor, the animals were inoculated with 25,000 B16-F10 melanoma cells in a 1:1 matigel solution at Day 0, subcutaneously in the right flank. Vaccine treatment or Control Treatment then began on day 7, at which time all mice had an identifiable tumor masses ˜100 mm³ in volume. Mice were randomly assigned into the following four (4) groups after 7 days. The date on which the animal was inoculated is considered Day 0. The respective treatment groups were provided the designated treatment on day 7, Day 12, and Day 17. The treatment groups were:
Control (Saline, n=16)
VetiVax™ (n=22)
RabiVax™ (n=7)
RabiVax™+Adjuvant (n=7)

### Preparation of the Tumor Tissue Vaccine Preparations:

The VetiVax™ and RabiVax™ Vaccines were prepared from tumor tissue obtained from donor mice. Mice in each treatment group did not undergo surgical debulking of an autologous tumor mass. The preparations for each group were prepared as follows.

### Control Group—Saline

VETIVAX GROUP—Each mouse received a volume of 250 μl of a tumor tissue cell preparation comprising 5e6 cells (tumor cells prepared from a donor mouse) combined with 1 mg SIS. This preparation was designated the VetiVax™ preparation. Mice were given three subcutaneous injections of the VetiVax™ preparation at day 7, day 12 and day 17 at the tail base. The volume of each VetiVax™ dose was approximately 250 μL.

RABIVAX GROUP—Each mouse receive about a total volume of 250 μL of the RabiVax™ preparation on day 7, 12 and 17. The RabiVax™ preparation was made up of 5e6 cells that had been incubated with a combination of the 3 epitope peptides, these peptides having been selected and isolated from epitope motifs of the rabies Pasteur strain protein sequence (See FIG. 4, Example 2).

To fixate the tumor tissue cell preparation with the epitope peptides, the preparation was combined with an amount of the 3 epitope peptides (Example 2), and incubated in 2.5% glutaraldehyde (v/v) at room temperature for 10 minutes (150 μg of combined peptides per 5e6 tumor cell, 2.5% glut.). After the incubation, the incubate was washed multiple times in PBS to remove the glut and any free peptide. centrifuged, and washed by suspending in PBS multiple times. For the last PBS wash, a final concentration of 150 μg of the combined 3 epitope peptides per 5e6 cell was obtained. The epitope peptide fixated 5e6 cell preparation was combined with 1 mg SIS to form the vaccine, RabiVax™. A total volume of 250 μl of the RabiVax™ was then injected into each animal on each of the treatment days 7, 12 and 17.

RABIVAX +Adjuvant GROUP—The RabiVax™ preparation described above was combined with an adjuvant. The adjuvant included was CpG1826 +MPLA, 10 μg each. The final concentration of the rabies peptide in the preparation was 150 μg rabies peptide per 5e6 tumor cells (ratio, scaled up) in a total volume of 250 μL. Animals were treated on day 7, 12, and 17 after the implant day (Day 0).

B16F10 Melanoma Anti-Tumor Efficacy. Individual tumor volume growth curves in response to each of the following 4 treatment regimens was assessed
1. PBS (Control) (were the Control animals vaccinated for rabies?)
2. VetiVax™
3. RabiVax™ (VetiVax™+Rabies antigen, cross-linked)
4. RabiVax™+Adjuvant

FIG. 2D provides a comparative analysis of the 3 treatment groups: (A) saline (n=16); (B) VetiVax™ (n=22), (C) RabiVax™ (n=14). These different tumor volume growth curves for B16-F10 tumors for each of the aforementioned treatments were statistically analyzed, Statistical comparisons are based on one-way ANOVA, followed by post hoc Tukey's pairwise comparisons. The asterisks denote statistical significance at the level of * p<0.05. ANOVA, analysis of variance.

### Example 4—Immuno-Memory Enhanced Cancer Vaccines

The present example demonstrates the utility of the present invention for providing a wide variety of immuno-memory enhanced anti-cancer and/or tumor vaccines employing methods that provide peptide and/or antigen associated with an infectious agent that the subject had been previously exposed to (immune memory).

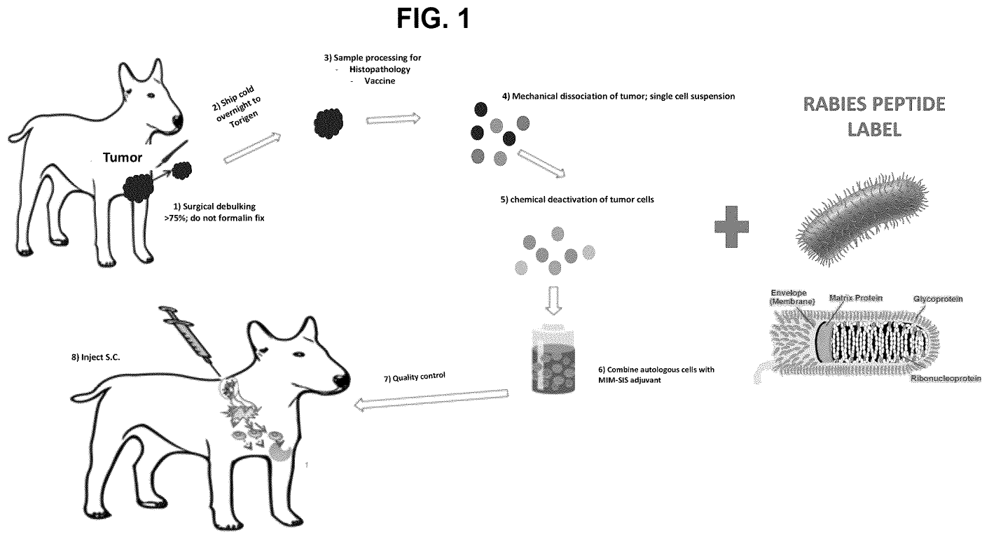

Utilizing the immunological memory response associated with traditional vaccines as a means to enhance immuno-response to a cancer antigen, a virus antigen/peptide of virtually any commonly administered viral vaccine is administered with the cancer and/or tumor antigen. For example, a cancer antigen associated with melanoma, may be administered with a rabies virus peptide, in a canine. A similar approach to that presented in FIG. 1 may be used in substituting the rabies virus peptide for another viral peptide, and a whole tumor tissue preparation may be substituted with a defined cancer antigen peptide/protein.

As described herein, a subject's immune memory mechanisms will serve to target cellular immune response against the viral peptide/protein component in the preparation, and as a result concomitantly provide an increase in cellular immune response to the cancer and/or tumor cell specific antigens included with the treatment preparation.

The tumor and/or cancer antigen component of the vaccine may be supplied in whole-tissue preparations (autologous, allogenic, xenogeneic), whole cells, a tumor cell lysate, vaccines, whole cell vaccines, a tumor and/or cancer associated antigen (single antigen, multiple antigens, whole cancer and/or tumor proteins), a neoantigen (single, multiple, whole protein, peptides), or any combination of these.

The virus antigen labeled cancer and/or tumor antigen preparations and formulations may be administered in any variety of delivery forms employing those techniques known to those of skill in the pharmaceutical arts, together with the teachings provided herein. By way of example, the vaccines may be formulated so as to be suitable for subcutaneous, intramuscular, intravascular, intra-tumoral (such as upon a resected tumor bed, or encased around a tumor), peritoneal, etc.

The following Table lists examples of various infectious pathogen based vaccines that are routinely administered to veterinary and human subjects. These provide suitable, non-limiting examples of an antigen that may be included and/or substituted or exchanged with those components of the immune memory enhanced preparations and treatments, formulations and products prepared therefrom.

TABLE 1
Antigen Candidates for Preparations
(Alone or in any combination)

Listeria spp.
Enter hemorrhagic E. coli
Bordetella pertussis
Brucella spp (mainly veterinary)
Haemophilus influenza
Salmonella spp
Shigella spp
Streptococcus pneumoniae

### Target Epitope Peptide Sequences and Associated Infectious Disease

Disease:

| Peptide | | | | | | | |
|---|---|---|---|---|---|---|---|
| Candi- | | | | | | | |
| date | | | | | | | |
| #/Se- | | | | | | | |
| quence | HPV* | Tetanus | Measles | Mumps | Rubella | Varicella* | Poliovirus* |
| 1 | RAHYNIV | QYIKANSKFIGI | KLWCRH | KPRTSTPV | GLLACSAKCL | ILIEGIFF | TTHIEQKA |
| | TFCCKCD | TEL | FCV | TEF (SEQ | YYLRGAIAPR | V (SEQ | LAQGLGQ |
| | S (SEQ ID | (SEQ ID | (SEQ ID | ID NO: 21) | (SEQ ID | ID NO: | (SEQ ID |
| | NO: 7) | NO: 11) | NO: 16) | (abun.) | NO: 27) | 33) | NO: 38) |
| | | | (CD8-B) | | | | |
| 2 | | NSVDDALINST | FMYMS | ALDQTDI | GFLSGVGPMR | NIQPGY | |
| | | KIYSYFPSV | MLLGV | RV (SEQ | LRHGADT | RSI (SEQ | |
| | | (SEQ ID | (SEQ ID | ID NO: 22) | (SEQ ID | ID NO: | |
| | | NO: 12) | NO: 17) | (sustained) | NO: 28) | 34) | |
| | | | (cyt) | | | | |
| 3 | | PGINGKAIHLV | ILPGQDL | | APPTLPQPPR | SLPRSR | |
| | | NNESSE (SEQ | QYV | | AHGQHYGHHH | TPII | |
| | | ID NO: 13) | (SEQ ID | | HQLPFLG | (SEQ ID | |
| | | | NO: 18) | | (SEQ ID | NO: 35) | |
| | | | (both) | | NO: 29) | | |
| 4 | | | | | TRWHRLLRMP | | |
| | | | | | VR | | |
| | | | | | (SEQ ID | | |
| | | | | | NO: 30) | | |
| | | | | | (C-area of | | |
| | | | | | the peptide) | | |
| Peptide | | | | | | | |
| Candi- | | | | | | | |
| date | | | Canine | Canine | | | |
| #/Se- | Canine | | Adenovirus- | para- | | | |
| quence | Parvovirus | Distemper | 2 | influenza | Hep A | Hep B | dTAP |
| 1 | QPDGGQP | QKTNFFNPNRE | CYIIGNC | NTVEYFT | ELDKWA | SSWAFA | FIGITELKK |
| | AVRNERA | FDFR | AVL | SQ (SEQ | (SEQ ID | RFLW | LESKINKV |
| | T, | (SEQ ID | (SEQ ID | ID NO: | NO: 31) | (SEQ ID | F (SEQ ID |
| | (SEQ ID | NO: 14) | NO: 19) | 23) | | NO: 36) | NO: 39) |
| | NO: 8) | | | | | | |
| 2 | GAVQPDG | | | GVTGLLT | | | |
| | GQPAV | | | NAA (SEQ | | | |
| | (SEQ ID | | | ID NO: 24) | | | |
| | NO: 9) | | | | | | |
| 3 | | | | IPNPLLGL | | | |
| | | | | D; (SEQ ID | | | |
| | | | | NO: 25) | | | |
| Peptide | | | | | | | |
| Candi- | Haemo- | | | | | | |
| date | philius | | | | | | |
| #/Se- | Influenzae | | | | | | |
| quence | b | Pneumococcal | Rotavirus | Influenza | Meningococcal | Tuberculosis | Covid-19 |
| 1 | GDKTTF | DRVPEEASR | DEMVR | GLFGAIA | PNTRYRTPN | NVTSIH | SFIEDLLFN |
| | KQS | (SEQ ID | ESQRN | GFIEGG | (SEQ ID | SLLD | KV (SEQ ID |
| | (SEQ ID | NO: 15) | G (SEQ | W (SEQ | NO: 32) | EGKQS | NO: 40) |
| | NO: 10) | | ID NO: | ID NO: | | LTKLA | |
| | | | 20) | 26) | | (SEQ ID | |
| | | | | | | NO: 37) | |

*= incomplete

### Target Epitope Proteins

| Disease | HPV* | Tetanus | Measles | Mumps | Rubella | Varicella* | Poliovirus* |
|---|---|---|---|---|---|---|---|
| 1 | The | ToxC | Measles | Haemagglutinin- | E1, and E2 | gE | VP4 |
| | Papillomavirus | | hemagglutinin | neuraminidase | | envelope | |
| | Major Capsid | | | | | protein | |
| | Protein L1 | | | | | | |

| | Canine | | Canine | | | | |
|---|---|---|---|---|---|---|---|
| Disease | Parvovirus | Distemper | Adenovirus-2 | Canine parainfluenza | Hep A | Hep B | dTAP |
| 1 | VP2 | Hemagglutinin | CAVpIX | P/V Proteins | LHBs | LHBs | FHA |
| 2 | | | | | MHBs | MHBs | Pertactin |
| 3 | | | | | SHBs | SHBs | FIM |

| | Haemophilius | | | | | | |
|---|---|---|---|---|---|---|---|
| Disease | Influenzae b | Pneumococcal | Rotavirus | Influenza | Meningococcal | TB | Covid 19 |
| 1 | PRP-T; | C- | VP6 | Influenza Virus | NHBA | E-sat6 | Spike |
| | | polysaccharide | | Hemagglutinin | | | Protein |
| 2 | | | | | fHbp | | G protein |

Canine parvovirus
Distemper (citation for a specific distemper antigen)
Canine adenovirus-2
Canine parainfluenza virus
Brucella spp

### Human

Hepatitis A
Hepatitis B
Diphtheria, tetanus and whooping cough
Hemophilus influenzae type b
Polio
Pneumococcal
Rotavirus
Influenza
Chickenpox
Measles, mumps, rubella
HPV
Meningococcal
Listeria spp.
Enterohemorrhagic E. coli
Bordetella pertussis
Brucella spp
Haemophilus influenzae
Salmonella spp
Shigella spp
Streptococcus pneumoniae
   The candidate disease-associated antigens listed here may be provided individually or in any combination.

TABLE 2
Cancer and/or Tumor Antigens
(alone or in any combination)

Whole-tissue-Autologous, allogeneic, xenogeneic
Whole cell-Autologous, allogeneic, xenogeneic
Tumor cell lysate-Autologous, allogeneic, xenogeneic
Tumor-associated antigen-single, multiple,
Whole protein, peptides
Neoantigens-single, multiple
Whole protein, peptides

TABLE 3
Adjuvants that may be included with
the formulations/preparations:

SIS: Particulate, gel, sheet
Alum
TLR agonists (i.e. CpG oligonucleotides,
Monophosphoryl lipid A, flagellin)
cGAMP associated derivatives
Poly(I:C)
Imiquimod

TABLE 4
Vaccine Memory Associated Adjuvants

Live-attenuate virus/bacteria
Heat (or) chemical inactivated virus/bacteria
Virus-like particle
Bacterial ghost
Protein
Peptide
Carbohydrate
DNA/RNA, specific to pathogen of interest

TABLE 5
Delivery Forms

MIM-SIS, as vehicle (particulate, sheet, gel)
Oil and water emulsion
Lipid formulation (i.e. liposomes)
Controlled release polymer-based formulation (i.e. PLGA)
Hydrogel

TABLE 6
Other combinations:

Cytokines (i.e. IL-12, GM-CSF, IL-2, IL-7, IL-15)
Immune check point blockade (i.e. anti-PD-L1, anti-CTLA4)
Antibody agonist (i.e. anti-OX40)

TABLE 7
Routes of administration,
including but not limited to-

Subcutaneous
Intratumoral
Intravenous
Intramuscular
Intranodal
Intradermal

The following Table presents a number of vaccines that are routine pathogen-based vaccinations for veterinary (canine, feline, equine, bovine) and human subjects. (*=CORE vaccines recommended for all; **=Non-CORE vaccines recommended based on risk factors).

TABLE 7
Canine (from the AAHA vaccination guidelines, 2017)
Mandatory: Rabies
CORE* vaccines: Distemper, Adenovirus-2, Parainfluenza, Parvovirus
Non-core**: Bordetella bronchiseptica, Leptospira (multiple serovars) Borrelia burgdorferi
(Lyme disease), canine influenza (H3N8, H3N2), Crotalus atrox (only in dogs with risk of
exposure), enteric coronavirus
Feline (from the AAFP vaccination guidelines, 2013)
Mandatory: Rabies (depends on jurisdiction)
CORE vaccines: Panleukopenia, herpesvirus-1, calicivirus, Feline leukemia (for kittens)
Non-core: Feline leukemia (for adults, depending on risks), feline immunodeficiency, Bordetella
bronchiseptica, feline infectious peritonitis (coronavirus),
Equine (from the AAEP guidelines)
Mandatory: Rabies (highly dependent on jurisdiction)
CORE vaccines: Eastern/Western/Venezuelan equine encephalomyelitis, Tetanus, West Nile virus
Non-core: Anthrax, Botulism, equine Influenza, equine herpesvirus, equine viral arteritis,
leptospirosis, Potomac horse fever, Streptococcus equi (strangles), snake bite
Bovine (varies beef cattle v. dairy cattle, life stage and geography). Rabies not mandatory in most
jurisdictions.
Beef herds: 7-Way Clostridial, infections bovine rhinotracheitis (IBR), bovine viral diarrhea
(BVD), parainfluenza-3 (PI-3), bovine respiratory syncytial virus (BRSV), Mannheimia
haemolytica, Histophilus somni, Campylobacter fetus, Trichomonas foetus, leptospirosis,
rotavirus, coronavirus, E. coli, Johnes disease, Moraxella bovis.
Humans
“Mandatory” Vaccines: MMRV, Polio, Hepatitis B, Meningitis, HPV, Influenza, Hepatitis A,
PPSV23, Hib, Rotavirus, tetanus

### Example 5—Soluble Immuno-Enhanced Vaccine Preparations

A non-antigen specific approach for providing an immune enhanced cancer vaccine is provided in the present example.

The same immunogenic materials associated with vaccines above could be delivered in a soluble form, conjugated to one or more carrier proteins (i.e. streptavidin, KLH, BSA, etc.), conjugated to or encapsulated by MIM-SIS or other extracellular matrix formulation, or produced as an alternative nano/micro formulation, such as encapsulation in alginate, chitosan, or other material, to be delivered by any variety of routes, including subcutaneous, intramuscular, intravenous, intratumoral, upon a resected tumor bed, or encased around a tumor.

### Example 6—Immuno-Active Transformation of a Tumor Site or Tumor (Cold to Hot Site Activation)

The delivery of the herein described preparations may be administered to a subject through an intra-tumoral administration directly in or on a tumor, and would facilitate the transformation of a “cold” (e.g., non-immuno-responsive) tumor into a “hot” (e.g., immuno-responsive) tumor. So transformed, the “hot” tumor site would recruit immune cells to that site, resulting in the penetration of B-cell and T-cell populations into the “hot” tumor site. The resulting “hot” (e.g., pro-inflammatory) tumor site environment would in this manner enhance and/or promote tumor clearance. Alternatively, any other route of administration may be employed as well (intramuscular, ip, etc.)
Intratumoral, not oncolytic viruses
   1. Intratumoral administration of seasonal flu vaccine (viral)
   2. Alpha-gal lipid intratumoral administration, in-situ autologous cancer vaccines (bacterial)
   3. Intratumoral administration of infectious disease vaccines, commentary piece
   3. Intratumoral administration, yellow fever vaccine
   5. Intratumoral administration, rotavirus vaccine
      Virus-specific T-cells can be exploited for cancer immunotherapy, model virus-like vesicles, intratumoral delivery of virus-specific peptides.

### Example 7—Adoptive Cell Therapy (CAR-T) Boosted by co-Administration with Viral Antigen

A significant challenge in the development of CAR-T therapies is the phenotype of interest for the T-cells to be reprogrammed. Specifically, T-cell stimulation to facilitate expansion of appropriate numbers of T-cells for the development of CAR-T therapies results in a high percentage of effector memory T-cells. These cells are less cytotoxic, more inflammatory and less capable of differentiating into long-lasting central memory T-cells. This could lead to sub-optimal therapies with short life spans of function in the body.

The strategy presented here employs vaccine-associated memory antigens to drive expansion of T-cells ex-vivo for the development of CAR-T therapies. The resulting phenotype of these cells would be more appropriate for translation to cancer immunotherapy.

### Co-Administration of CAR-T with Vaccine-Associated Antigen

The co-administration of vaccine-associated antigens with traditional CAR-T cells directed against tumor-associated antigens has no clear mechanism for improved efficacy. However, here is presented the development of vaccine associated antigen specific CAR-T cells (rabies-specific) with co-delivery of tumor cell labeling a component. In other words, intratumoral injection of a rabies-peptide modified lipid that inserts into tumors and labels them followed by delivery of a rabies-specific CAR-T therapy that would identify these labeled cells and kill the labeled tumor cells accordingly. In this way, CAR-T therapy is provided that is tumor type agnostic.

CAR T Cell therapy: improvements through harnessing immune memory. Chimeric antigen receptor T cells are T cells taken from the patient that are then transfected with a viral vector to express a synthetic antigen receptor on the surface. This antigen receptor can be programmed to present a vast array of tumor specific antigens. Such would provide an autologous leukocyte stimulation therapy. The advantages of this approach include:
Great clinical efficacy, and minimal side effects
Reduction of cytokine release syndrome (CRS), a barrier to its efficacy, as treatments for it result in reduced treatment efficacy.

CAR T Cell Therapy within immunological memory. One of the problems with CAR-T cell therapy is T cell persistence. T effector cell formation is forced with the inoculation of the antigen receptor, but these cells appear to struggle to differentiate into memory T cells. Essentially, CAR T cell therapy is only as effective as the T Effector cells remain in circulation, without self-renewal they eventually die off and don't leave any memory cells behind. The cancer cell memory cells remaining are not the T effector cells from CAR, but memory T cells left behind from tumor antigens.

Long term memory (or central memory) T cells (Tcm) have shown increased cytotoxic efficacy against cancer cells, and the ability to self-renew after Car T cell inoculation. These T cells are expressed in increasing numbers based upon the time after infection. Over time they also express less phenotypic heterogeneity and “bystander activation” (an important component of cytokine release syndrome).

Effector T cells from central memory (long term memory T cells) are more cytotoxic, more proliferative, and more persistent than their short term (Tem) counterparts.

Tem cells are less prone to bystander activation, and therefore will result in a lower incidence of the main side effect of CAR T cell therapy (CRS).

The ability to stimulate T cells with peptides prior to CAR transfection will result in the proliferation of Tem cells for that specific peptide. This would result in a higher proportion of Tcm in the administered CAR, and therefore higher efficacy with less incidence of CRS.

### Example 8—Infectious Disease-Other Formulations without Co-Administration with a Disease-Associated Antigen—Naturally Acquired and/or Acquired Immunity Model Preparations/Formulations

In some populations, subjects will not routinely receive vaccinations against common viral pathogens. In these subject populations, immune response is governed by acquired immunity. For example, immunity to common infectious agents for the particular population might include rhinovirus, varicella-zoster virus, and influenza. Antigenic moieties of such agents would be employed as anti-tumor enhancing components of vaccines for tumor immunotherapy or immunoprophylaxis.

The present example demonstrates the use of a subjects naturally developed acquired immunity in enhancing immuno-response to a defined cancer antigen, viral antigen or bacterial antigen.

Disease states that are potentially cured or treated without vaccination, yet still yield immunological memory, may be manipulated according to the methods described herein. Examples of the vaccines that may be developed in a subject having an acquired immunity (and not having had a specific vaccination for a conventional viral pathogen) may be prepared to treat the exemplary infections listed here, or alternatively to an identified cancer and/or tumor. These infections are exemplary only, as virtually any infection may be treated according to the present example employing the teachings disclosed herein. These vaccines are particularly well suited for use in countries without wide-spread vaccination programs typical in developed countries. In these preparations, a target viral antigen (such as a SARS-COV-2 spike protein) would be coupled with a viral antigen that the subject had developed an acquired immunity against (for example, a flu virus antigen) or a viral antigen to which a subject had developed an antibody response to as a result of vaccination (such as rabies).

These preparations could be formulated in an identical fashion as vaccination dependent antigens. Prior testing would be conducted to validate acquired immune memory against said infection (i.e. neutralizing antibody to SARS-COV-2). Alternatively, precedent in experimental models could validate that highly conserved antigens could be used without presence of true immunological memory; for example, influenza specific hemagglutinin.

The discase of interest would determine the target demographic. Namely, the prevalence of influenza, the common cold and certain venereal diseases in the developed world as compared to the chronic exposure to disease of poverty, such as tuberculosis, malaria and HIV/AIDS.

### Examples (Relevant Antigens of Interest)

COVID-19, SARS-COV-2
Influenza
Common cold (i.e. rhinovirus, coronavirus)
Streptococcus infection
Staphylococcus infection
Epstein-Barr virus (mono)
Herpes simplex virus
Varicella zoster virus
Prions
Parasites

Additional candidates for acquired immunity applications:

Human Herpesvirus 6 and 7—Antibodies to this virus are present in almost everyone by age 5.

Rhinovirus C—90% of children hospitalized with acute asthma attacks were shown to have detectable HRV.

Human Adenovirus C—The common species C adenoviruses (serotypes Ad1, Ad2, Ad5, and Ad6) infect more than 80% of the human population early in life

Human Respiratory Syncytial virus. hRSV is associated with a rate of infection close to 34 million children under 5 years old per year. Specifically, hRSV is responsible of nearly 63% of total ALTRI cases and between 19 to 81% of the total viral infections affecting the lower respiratory tract in children.

Third-world Diseases: Tuberculosis, Malaria, Lyme disease, HIV/AIDS

### Rabies Virus Peptides

(aa: 241-255)
(SEQ ID NO: 4)
ACKLKLCGVLGLRLM
(aa: 349-363)
(SEQ ID NO: 5)
KSVRTWNEIIPSKGC
(aa: 270-284)
(SEQ ID NO: 6)
WCPPGQLVNLHDFRS

### Example 9—Epitopic Peptide Selection Protocol

Peptide sequences were taken from the immunogenic regions of surface proteins on viral particles. These correspond with “B cell epitopes”, or targets that are primarily neutralized by antibody production from B cells. In viruses without defined B cell epitopes in literature, conserved sequences from better studied viruses were used instead being conserved across similar viruses indicates the potential for similar immunogenicity

Immune “Noise”—the selected antigenic components and/or antigens of the preparations provide an improved correction to reduce and/or otherwise accommodate immunc interference. “Noise” is a term used to describe the immune system's ability to filter out “self” tissue from immunogenic “other” tissue. An autologous cancer vaccine by design creates “noise” as self-tissue is inevitably mixed in with tumor tissue. Central memory T cells are less prone to immunological exhaustion and self-tolerance. Tem cells are still affected by immunological noise, but not as dramatically as naive acutely stimulated T cells. Theoretically, the Tem cells should be less prone to being affected by immunological noise, and their activation should result in increased cytotoxicity.

IgG antibodies are more prone to opsonization than IgM antibodies. IgG Fc binds specifically to phagocytes, increasing antigen uptake and therefore presentation. Therefore, memory B cell responses should be more apt to cause increased opsonization and cross presentation, further increasing neoantigen presentation and uptake. The tumor cells being “tagged” by IgG may reduce “self” uptake and presentation, reducing the proportion of anergic lymphocytes. Increased cross presentation and opsonization would therefore benefit by reducing the chance that “noise” from tissue would induce self-tolerance and prevent vaccine efficacy.

The peptides listed below represent 5 amino acid overlapping 15-mer from the rabies virus glycoprotein extracellular domain that have been reported to have B-cell epitope content. These peptides were screened for use as peptide candidates for inclusion in an autologous cancer vaccine combination preparation. In this study, canine serum was used from clinical candidates and an ELISA test was run to determine immunoreactivity of rabies-specific antibodies in canine scrum to each individual peptide candidate. From this study, a high level of variability in immunoreactivity between the peptides and individual clinical patient samples was revealed across the clinical patient population. From this information, immuno-invoking preparations comprising a combination of peptides were defined. While the individual peptides have overlapping immuno-reactivity, use of a combination of two or more of these peptides in a preparation will provide an improved immuno-invoking antigenic component with effectivity across a broader, more heterogeneous, patient population.

Table of Candidate Peptides-
Screening Panel

| | |
|---|---|
| 1. | IFTNSRGKRASKGSE (SEQ ID NO: 41) |
| 2. | SKGSETCGFVDERGL (SEQ ID NO: 42 |
| 3. | DERGLYKSLKGACKL (SEQ ID NO: 43) |
| 4. | GACKLKLCGVLGLRL (SEQ ID NO: 44) |
| 5. | LGLRLMDGTWVAMQT (SEQ ID NO: 45) |
| 6. | VAMQTSNETKWCPPG (SEQ ID NO: 46) |
| 7. | WCPPGQLVNLHDERS (SEQ ID NO: 6) |
| 8. | HDFRSDEIEHLVVEE (SEQ ID NO: 47) |
| 9. | LVVEELVKKREECLD (SEQ ID NO: 48) |
| 10. | ENPRLGMSCDIFTNS (SEQ ID NO: 49) |

### Example 10—Infectious Disease; COVID-19 Fusion Protein Example

SARS-COV-2 spike protein will be fused (recombinant, or crosslinking chemistry) with the immunogenic sequence(s) from the rabies virus to create more immunological reactive compound that could promote higher titer antibody responses driving protection.
1) Protein-protein
2) Protein-peptide
3) Protein-carbohydrate
4) Peptide-peptide
5) Peptide-carbohydrate

### Example 11—Infectious Disease Applications

Infectious Disease protocol-In the present example, the recombinant proteins were purchased from external vendors. These recombinant proteins were modified via amine chemistry to become thiol reactive; specifically, recombinant proteins were activated with Sulfo-LC-SPDP (˜10 mole SPDP per mole protein). Sulfo-LC-SPDP is a heterobifunctional crosslinker that reacts primarily with amines on protein surfaces, and will subsequently react with free thiols (cysteine residues) on rabies specific peptide sequences. Rabies peptides are crudely mix by physical mixture and incubated at room temperature for 2 hours prior to vaccination of research mice—no additional purification was performed at this time. Mice (n=5) will be vaccinated initially with 25 μg of target protein of interest and boosted twice subsequently with 12.5 μg each at three-week intervals. Bleeding will be performed to determine antibody titer and ex-vivo analysis of relevant immune cell populations will be performed at the termination of the study (8-weeks).

### Treatment Groups

### COVID-19

1. Saline
2. SARS-COV-2 spike protein, alone
3. SARS-COV-2 spike protein, alum
4. SARS-COV-2 spike protein, rabies
5. SARS-COV-2 spike protein, rabies+alum

### Tuberculosis

1. Saline
2. ESAT-6 protein, alone
3. ESAT-6 protein, alum
4. ESAT-6 protein, rabies
5. ESAT-6 protein, rabies+alum
   Dose: 25 μg+12.5 μg (3-weeks)+12.5 μg (6-weeks)

The treatments are expected to provide an enhanced anti-infectious agent result in the treated subject, compared to a subject having a disease associated with the infectious agent not receiving the treatment or preparation as described here.

### Example 12—Tumor Reduction and Survival Benefit—Prior Vaccination to Rabies

The present example demonstrates that an improvement in survival and a reduction in tumor size resulted in animals first vaccinated for rabies, compared to animals that have not been treated with rabies. The data is shown in FIG. 5.

The present example also elucidates the mechanism of action for the described invention. Specifically, anti-tumor efficacy was demonstrated to be specific to vaccine associated memory response to rabies antigens covalently decorating autologous tumor tissue vaccines.

To explore the dependency of therapeutic efficacy on rabies vaccine memory to prior, a tumor challenge was administered to C57BL/6J mice that had or had not been vaccinated against rabies. These animals were subsequently treated with (1) autologous tumor tissue vaccines with rabies-specific peptides conjugated to cell surface or (2) with the rabies-specific peptides mixed with (not affixed to) autologous tumor tissue vaccine as a physical mixture. Tumor challenge models and therapeutic regimen were repeated as laid out in Example 2, with the exception that therapy was initiated at day 12. Only one group of animals did not receive vaccination with rabies.

The results of the study revealed that therapeutic anti-tumor efficacy was dependent on vaccine memory. Specifically, tumor challenge in C57BL/6J mice who had not been vaccinated against rabies resulted the development of larger tumor volumes (3.2-fold increase; p<0.05) and a reduction of median survival time (20 days vs. 25.5 days; p<0.05), as compared C57BL/6J mice who were vaccinated against rabies prior to tumor challenge. Additionally, a 45% reduction in tumor volume evidences that a preparation wherein the rabies peptide is conjugation to the autologous tumor cells improves anti-tumor efficacy compared to tumor volume reduction upon treatment with a physical mixture of the rabies peptides with the tumor tissue vaccine. While not intending to be limited to any specific mechanism of action or theory, the co-localization of rabies peptides onto the tumor cells provides an improved vaccine associated memory response in the animal, and is therefore optimal for therapeutic functionality.

### Example 13—Prophylactic Preparations-RabiVax Mechanism of Action

The present example presents data concerning the mechanism of action by which the rabies vaccine preparations function mechanistically.

The present example presents the utility of the present preparations and methods for the tumor-type agnostic prophylactic prevention of cancer. Specifically, prophylactic anti-tumor efficacy was assessed for B16F10 melanoma as well as CT26 colon cancer.

Rabies vaccinated mice were treated via subcutaneous tail base administration three times at weekly intervals prior to tumor challenge in a subcutaneous flank model. Per dose, mice received 5e6 total B16F10 cells, 150 μg rabies-specific peptide adjuvant and 1 mg MIM-SIS. For CT26 colon cancer, 5e6 cells were utilized for the tumor challenge; For B16F10 melanoma, again, 2.5e4 cells were employed for the tumor challenge in order to compare results to previous established therapeutic models.

The results of this study demonstrate that treatment with autologous tumor tissue vaccines (CT26 colon cancer or B16F10 melanoma) with rabies-specific peptides conjugated to a cell surface resulted in significant delay in tumor growth, as well as an enhancement in reduction/inhibition of tumor development and tumor size growth (mean tumor volume). Notably, a reduction in tumor growth by 4.4-fold (p<0.01) and 64.6-fold (p<0.01) as compared to the administration of saline for CT26 colon cancer and B16-F10 melanoma, respectively, was observed. In addition, the present example and in vivo data results demonstrate that the methods and preparations provided and described here are not limited to a particular cancer type or tumor type, and are in fact effective against a number of different tumor types.

### Example 14—Tumor Growth In Vivo-Vaccination with VetiVax™, RabiVax™, or SuperAg Vax RabiVax™

The present example demonstrates the effectiveness of three vaccine formulations, for inhibiting tumor growth in vivo, and the population range of efficacy of each of the three vaccine formulations.

The data collected from this study are presented in FIG. 8.

Tumor Volume in Tumor-Bearing Subjects-Day—20 post Inoculation with vaccine formulations, VetiVax™, RabiVax™, or SuperAg Vax RabiVax™. The scatter plots illustrate tumor volume pooled sample data sets from tumor bearing subjects treated with one of the three vaccine formulations. The RabiVax™ (B-cell epitopes) vaccine formulation comprises three B-cell epitope peptides derived from the Rabies Virus glycoprotein (a cocktail of 3 B-cell epitope peptides), but does not contain T-cell epitopes derived from Rabies Virus glycoprotein and nucleoproteins. SuperAg Vax RabiVax™ (B-cell+CD4 T-cell) comprises the three B-cell epitope peptides (cocktail of 3 B-cell epitope peptides) and Helper T-cell epitopes derived from the Rabies Virus glycoprotein and nucleoproteins. The VetiVax™ vaccine formulation is previously described herein. All subject animals had been previously Rabies-vaccinated. These Rabies-vaccinated mice were inoculated with 25,000 B16F10 melanoma cells SQ flank on day 0 and then monitored for tumor growth progression by calipers at defined intervals. The data plots represent the tumor volume in these subjects at t=20 days, pooled across multiple studies performed under identical experimental conditions. From this data, the SuperAg Vax™ vaccine formulation treatment did not significantly reduce the mean tumor volume in the subject animal population compared to the tumor size in the subject animal populations receiving the RabiVax™ vaccine formulation treatment or subject animals receiving the VetiVax™ vaccine formulation treatment.

The data does demonstrate that the SuperAg Vax™ vaccine formulation treatment resulted in a wider range of effectiveness among a greater number of tumor-bearing subject animals in the populations examined, and did significantly eliminate the likelihood of outlier non-responder tumor-bearing animals. This data demonstrates the additional benefit of the combination antigen vaccine, SuperAg Vax™, containing B-cell and helper T-cell epitopes, compared to B-cell epitope alone vaccine formulations RabiVax™, as well as compared to the VetiVax™ formulation receiving subject results, across a wider population of tumor-bearing subjects in vivo.

### Example 15—Therapeutic and Prophylactic Efficacy of Immuno-Memory Autologous Therapy

The efficacy of the RabiVax™ and SuperAg Vax™ vaccine formulations was compared in FIG. 9A as a therapeutic, and in FIG. 9B as a prophylactic for inhibiting/preventing tumor growth, in vivo.

FIG. 9A—For the therapeutic model, Rabies-vaccinated mice were inoculated with 25,000 B16F10 melanoma cells SQ flank on day 0, and then monitored for tumor growth progression by calipers at defined intervals. The subject animals were treated with either the RabiVax™ (triangle Δ data line) vaccine formulation or the SuperAg Vax™ (circle · data line) vaccine formulation. FIG. 9 B—For the preventative or prophylactic model, Rabies-vaccinated mice were vaccinated with the RabiVax™ or the SuperAg Vax formulation vaccine, once per weck for three weeks (day 0, 7 and 14), and then inoculated with 1,000,0000 B16F10 melanoma cells SQ flank on day 21.

The subject animals were treated with either the Rabi Vax™ (triangle data line) vaccine formulation or the SuperAg Vax™ (circle data line) vaccine formulation. A significant reduction in tumor size in animals previously treated with the SuperAg Vax™ vaccine preparation was observed, compared to tumor size in subject animals that had been pretreated with RabiVax™. The reduction in observed tumor growth is demonstrated beginning at about 12-14 days. The data demonstrates no statistically significant additional advantage or improvement upon administration of the SuperAg Vax™ formulation in a therapeutic setting (FIG. 9A), but additional benefit in a preventative setting (FIG. 9B).

### Example 16—Modification of Dosing-Interval Vaccine Boost Regimen on Efficacy of Immuno-Memory Autologous Vaccine Administration—RabiVax™ Vaccine Formulation Treatment

The variable efficacy of inhibiting tumor growth in an animal with the RabiVax™ vaccine formulation as a factor of varying the dosing interval with the vaccine formulation, is illustrated in the present example.

The results of the present study are provided at FIG. 10.

Rabies-vaccinated mice were treated with the vaccine formulation RabiVax™ as a single dose or as a regimen of multiple doses (Wide Boost (30 days) or Tight Boost (7 days) dosing schedule). The Tight Boost dosing regimen consisted of administering to each animal 3 doses of the RabiVax™ vaccine formulation on a 7-day boost dosing interval schedule (day 0, day 7 and day 14) (FIG. 10, top bar line data graph). The Wide Boost dosing regimen consisted of administering to each animal 3 doses of the RabiVax™ vaccine formulation on a 30-day boost dosing interval schedule (day 0, day 30 and day 60) (FIG. 10, lowest bar line data graph). One group of the subject animals received a single dose of the RabiVax™ vaccination formulation (FIG. 10, middle bar line data graph).

All treated subject mice were inoculated with 1,000,000 B16F10 melanoma cells, SQ flank on day 0, and then monitored for tumor growth progression by calipers at defined intervals.

The data demonstrates a less effective anti-tumor growth affect (a therapeutic decline) of the Rabi Vax™ vaccine formulation in animals treated according to the Tight 7-day boost dosing regimen. While not intending to be limited to any particular mechanism of action, it is contemplated that this observation may have resulted due to immune exhaustion or other factors. The anti-tumor growth effectiveness of a single dose of the RabiVax™ vaccine formulation was observed to not be inferior to the Wide Boost dosing regimen treatment. This data provides validation for a single dose model.

### Example 17—SARS-Cov-2 S1 Spike Protein Antigen Vaccination Formulations

Rabies-vaccinated mice (n=5) were treated as described herein. These rabies vaccinated mice were subsequently vaccinated (prime-boost) with S1 spike protein of SARS-CoV-2 either: (1) SARS-Cov-2 alone, (2) SARS-COV-2 chemically cross-linked/conjugated to Rabies virus glycoprotein B-cell epitope peptides or (3) SARS-COV-2 physically mixed with Alum. Blood was collected from all mice via submandibular puncture at weekly intervals. S1 spike protein specific anti-IgG was determined by ELISA from blood from each group collected two-weeks post-boost (week 5).

The results from the stud are presented in FIG. 11.

### Example 18—Identification of Particular Sequences for Vaccine Preparations

The present example provides specific sub-groups of sequences to be employed in various embodiments of the vaccine preparations.

In numerous instances, the sequence information provided below was/is related to a larger/longer sequence of amino acids, and may further include modifications/substitutions in the sequence that differentiates the sequence from native, naturally occurring segments of sequences presented here (for example, a smaller/shorter sequence may be part of a much larger and/or longer or complex sequence, and include one or more modified amino acid residues distinguishing it from the longer and/or more complex native sequence.).

Novel fusion proteins and/or peptides may be prepared with one or more of the sequences presented here.

The sequences designated in the following listing may be incorporated into the vaccine formulations of the invention. One or more of these sequences may be included as candidates in the preparations of the present vaccines. These may be employed as candidates to be used as “pre-existing” immunostimulatory element(s). As such, it is envisioned that these sequences may be included together with a “de-novo” element in the vaccine preparations disclosed.

Relevant sequences:
Distemper; B-cell epitope;
(SEQ ID NO: 54)
SHGMGIVAGSTGATQSAL;
The epitope;
(SEQ ID NO: 55)
MTAAQITAGIALHQSNLN
Parvovirus; B-cell epitope;
(SEQ ID NO: 56)
MSDGAVQPDGGQPAVRNER[[.]]
Adenovirus; B-cell epitope;
(SEQ ID NO: 57)
GWSFTRIKQRETPALGSP
Alternative Sequences;
Canine Distemper Virus:
(SEQ ID NO: 58)
SHGMGIVAGSTN,
(SEQ ID NO: 59)
GPSVSAENVRQ,
(SEQ ID NO: 60)
INPELRPIIGR,
(SEQ ID NO: 61)
TQSALKSTG,
(SEQ ID NO: 62)
MTAAQITAGIALHQSNLN,
(SEQ ID NO: 63)
GDKYPIHENDER,
(SEQ ID NO: 64)
QKTNFFNPNREFDFR,
(SEQ ID NO: 65)
ARGDIFPPY,
(SEQ ID NO: 66)
MSDGAVQPDGGQPAVRNERATG,
(SEQ ID NO: 67)
KTAVNGNMALDDTHA,
(SEQ ID NO: 68)
ALGLPPFLNSLPQSEGGTNFGYIGVQQ,
Canine Parovirus:
(SEQ ID NO: 69)
PKIFINLAKKKKAG;
(SEQ ID NO: 70)
GQPAVRNERATGS,
(SEQ ID NO: 71)
DGAVQPDGGQPAVRNER,
(SEQ ID NO: 72)
NSLPQSEGATNFGDIGVP,
(SEQ ID NO: 73)
GKRNTVLFHGPASTKGK

While the present disclosure makes reference to specific exemplary embodiments, the present disclosure may also be embodied or implemented in other specific forms without departing from its spirit or essential characteristics. Accordingly, the disclosed embodiments are to be considered in all respects only as illustrative and not restrictive. For instance, various substitutions, alterations, and/or modifications of the inventive features described and/or illustrated herein, and additional applications of the principles described and/or illustrated herein, which would occur to one skilled in the relevant art and having possession of this disclosure, can be made to the described and/or illustrated embodiments without departing from the spirit and scope of the disclosure. Such substitutions, alterations, and/or modifications are to be considered within the scope of this disclosure.

The scope of the invention is indicated by the appended claims rather than by the foregoing description of the present disclosure. The limitations recited in the claims are to be interpreted broadly based on the language employed in the claims and not limited to specific examples described in the present disclosure, including the detailed description, which examples are to be construed as non-exclusive and non-exhaustive. All changes which come within the meaning and range of equivalency of the claims are to be embraced within their scope.

It will also be appreciated that various features of certain embodiments can be compatible with, combined with, included in, and/or incorporated into other embodiments of the present disclosure. For instance, systems, methods, and/or products according to certain embodiments of the present disclosure may include, incorporate, or otherwise comprise features described in other embodiments disclosed and/or described herein. Thus, disclosure of certain features relative to a specific embodiment of the present disclosure should not be construed as limiting application or inclusion of said features to the specific embodiment.

In addition, unless a feature is described as being required in a particular embodiment, features described in the various embodiments can be optional and may not be included in other embodiments of the present disclosure. Moreover, unless a feature is described as requiring another feature in combination therewith, any feature herein may be combined with any other feature of a same or different embodiment disclosed herein. It will be appreciated that while features may be optional in certain embodiments, when features are included in such embodiments, they can be required to have a specific configuration as described in the present disclosure.

Likewise, any steps recited in any method or process described herein and/or recited in the claims can be executed in any suitable order and are not necessarily limited to the order described and/or recited, unless otherwise stated (explicitly or implicitly). Such steps can, however, also be required to be performed in a specific order or any suitable order in certain embodiments of the present disclosure. Furthermore, various well-known aspects of illustrative systems, methods, products, and the like are not described herein in particular detail in order to avoid obscuring aspects of the example embodiments. Such aspects are, however, also contemplated herein.

1. An immune memory enhanced preparation comprising:
a first component comprising a target moiety of interest; and
a second component that invokes an immune memory response in a subject, wherein the immune memory response to the second component in the subject enhances immune response to the target moiety compared to immune response to the target moiety in the absence of the second component.

2. The immune memory enhanced preparation of claim 1 wherein the target moiety of interest comprises a tumor antigen, a cancer antigen, a bacterial antigen, a viral antigen, a parasitic organism antigen, a fungal antigen, other infectious agent antigen, or a combination thereof.

3. The immune memory enhanced preparation of claim 1 wherein the second component comprises a peptide, protein, peptide or protein epitope, peptide or protein fragment of or associated with an infectious organism, a virus, a bacteria, a parasitic organism, a synthetic or naturally occurring immune invoking moiety, or any combination thereof.

4. The immune memory enhanced preparation of claim 3 wherein the peptide epitope of the second component comprises a viral epitopic peptide, a bacterial epitopic peptide, a parasite epitopic peptide, or any combination thereof.

5. The immune memory enhanced preparation of claim 3, wherein the peptide epitope of the second component comprises a peptide epitope having immuno-invoking specificity for a tuberculosis, rabies, tetanus, measles, distemper, polio, influenza, measles, mumps, rubella, chickenpox, diphtheria, hepatitis A or B, pneumonia, human papilloma virus, distemper, parvovirus, SARS, COVID, swine flu, Plasmodium, yellow fever, or parvovirus causing infectious agent.

6. The immune memory enhanced preparation of claim 1 wherein the subject is a veterinary subject or a human subject.

7. The immune memory enhanced preparation of claim 6 wherein the veterinary subject is a canine, feline, bovine, poultry, marine, avian, invertebrate, reptile or equine veterinary subject.

8. The immune memory enhanced preparation of claim 4 wherein the second component comprises a virus and the peptide epitope of the second component comprises a B-cell, T-cell or combination of B-cell and T-cell epitopes.

9. The immune memory enhanced preparation of claim 1 wherein the immune memory response is an acquired immune response in the subject.

10. The immune memory enhanced preparation of claim 1 wherein the second component comprises a fusion peptide, said fusion peptide comprising:
an N-terminal KKKGGG flanker sequence fused to a peptide of interest, wherein the peptide of interest comprises:
(SEQ ID NO: 4)
ACKLKLCGVLGLRLM;
(SEQ ID NO: 5)
KSVRTWNEIIPSKGC;
or
(SEQ ID NO: 6)
WCPPGQLVNLHDFRS,
or any combination thereof.

11. The immune memory enhanced preparation of claim 1 comprising an adjuvant.

12. The immune memory enhanced preparation of claim 11 wherein the adjuvant is CpG1826 and MPLA.

13. The immune memory enhanced preparation of claim 1 wherein the target moiety of interest comprises a cancer antigen or peptide or a tumor antigen or peptide.

14. The immune memory enhanced preparation of claim 1 wherein the first component and the second component are:
combined in a mixture;
fixed to each other;
covalently linked; or
conjugated to each other.

15. The immune memory enhanced preparation of claim 1 wherein the first component and the second component are fixed by glutaraldehyde fixation.

16. An anti-viral immune memory enhanced pharmaceutical preparation comprising:
a first component comprising a viral antigen of interest; and
a second component comprising a protein, peptide, carbohydrate, or combination thereof, wherein the second component invokes an immune memory response to the first component, and enhances immune response in a subject to the viral antigen of interest compared to immune response in the subject to the viral antigen of interest in the absence of the second component.

17. A method for inhibiting and/or preventing tumor growth in a subject comprising:
providing a vaccine formulation to the subject, the vaccine formulation comprising:
a first component comprising a tumor antigen characteristic of a tumor tissue harvested from the subject; and
a second component comprising a protein, peptide, carbohydrate, or combination thereof, the second component enhancing the immune response in the subject to the tumor antigen, and invoking an immune memory response; and
inhibiting and/or preventing tumor growth in the subject, wherein inhibition and/or prevention of tumor growth in the subject provided the vaccine formulation is enhanced compared to inhibition and/or prevention of tumor growth in the subject provided a vaccine that does not include the second component.

18. The method of claim 17 wherein the preparation is a soluble preparation and comprises a carrier comprising streptavidin, KLH, BSA or an extracellular matrix material.
